(19) United States
(12) Patent Application Publication
(10) Pub. No.: US 2022/0301666 A1
(43) Pub. Date: Sep. 22, 2022

(54) SYSTEM AND METHODS OF MONITORING A PATIENT AND DOCUMENTING TREATMENT

(71) Applicant: Bio1 Systems, Inc., San Carlos, CA (US)

(72) Inventors: Lauren SHLUZAS, San Carlos, CA (US); Alan SHLUZAS, San Carlos, CA (US); Gabriel ALDAZ, Palo Alto, CA (US); Miguel GARCIA, San Carlos, CA (US)

(21) Appl. No.: 17/701,231

(22) Filed: Mar. 22, 2022

Related U.S. Application Data
(60) Provisional application No. 63/163,934, filed on Mar. 22, 2021.

(51) Int. Cl. G16H 10/60; G16H 50/20; G16H 50/30; G16H 40/67; G16H 80/00; G06N 3/04; A61B 5/00; G06F 3/01

(52) U.S. Cl. CPC G16H 10/60; G16H 50/20; G16H 50/30; G16H 40/67; G16H 80/00; G06N 3/04; A61B 5/7264; G06F 3/015

(57) ABSTRACT

This disclosure provides an efficient, hands-free system and method for capturing and recording patient treatment and physiological data in critical care environments. The systems and methods described herein enables clinicians to record and transcribe patient information and physiological data onto an individual disposable medical record tag, which accompanies the patientthroughout initial stabilization and presentation to a treatment center. The data tag digitally stores a patient's health status, and displays a specific color based on a patient's degree of injury or if treatment is required. The data tag forms the center of a patient centric network PCN of connectedhealth devices. An artificial intelligence machine learning model is used in combination with predictive analytics to assess a patient's condition and provide clinical decision support for clinicians based on predictive analytical models.

### CROSS REFERENCE TO RELATED APPLICATIONS

This application claims the benefit of U.S. Provisional Patent Application No. 63/163,934, filed Mar. 22, 2021, titled “SYSTEM AND METHODS OF MONITORING A PATIENT AND DOCUMENTING TREATMENT”, the contents of which are incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

Additionally, this patent references U.S. patent application Ser. No. 15/79,5035 SYSTEM AND METHODS OF IMPROVED HUMAN MACHINE INTERFACE FOR DATA ENTRY INTO ELECTRONIC HEALTH RECORDS. All referenced patents are incorporated by reference.

### FIELD

This application relates generally to the documentation of medical treatment of a patient by a care provider in an electronic medium via data input systems and methods utilizing physiological monitoring systems, voice to text software, and automatic object detection and classification. Additionally, the incorporation of computer machine learning artificial intelligence provides the ability for automated patient monitoring and identification of changes in patient vital signs that warrant further clinical intervention.

### BACKGROUND

The Electronic Health Record (EHR) has revolutionized the health environment, providing near real-time documentation and immediate recall of a patient's entire clinical care and medical history. In controlled environments, such as primary care settings, the EHR has tremendous value. However in acute, uncontrolled, and non-traditional environments—e.g., surgery, rural/remote settings, emergency/trauma departments, and battlefields—the EHR is constrained due to its limited flexibility, non-intuitive work flows, menu-driven charting, reliance on robust communication connections, and dependence on manual data entry. Instead of aiding care, the EHR becomes a handicap, limiting the clinician's ability to provide hands-on clinical care. As such, there is an urgent need within healthcare settings to improve the interface and reduce the amount of time that clinicians spend interacting with the EHR; this is necessary to increase direct patient engagement and improve treatment outcomes.

Current practices in providing tactical field care and completing a Tactical Combat Casualty Care (TCCC) card affixed to a patient require that a lead medic provides care while a second acts as a scribe; recording information while following treatment guidelines. In this scenario, the lead medic may be distracted while communicating with his counterpart, while the second medic's skills are underutilized. Furthermore, documentation is at risk of error and loss during transfer to the military's MC4 electronic health record system. To improve military combat scenarios, there is a need to: 1) Reduce the number of medics per patient through hands-free, single-user data entry; 2) Incorporate an efficient data recording method to capture accurate information with reduced chance of human error; 3) Provide a streamlined solution that provides EHR continuity across disconnected groups, through digitally linking TCCC data to the MC4, with a robust solution for areas lacking internet connectivity.

Similar to the military environment, civilian first responders are typically disconnected from the local hospitals that receive their patients. In an emergency care situation, the first medical personnel to come into contact with a patient will initiate treatment; this includes an emergency medical technician (EMT), fire rescue, or emergency staff on presentation to an emergency department. Patient stabilization is the priority in these initial minutes with any care-related data being captured by whatever means available (often by writing on the backside of a latex glove). As the EHR is incompatible in acute/uncontrolled/non-traditional environments, documentation is often performed after the patient is stabilized, with clinicians relying on hand-written notes, verbal dictation, or memory to transfer information into the patient's EHR.

In larger mass casualty scenarios, limitations with the EHR are compounded. Due to the inability to log, treat, and track numerous patients that present in mass casualty events, patients are labeled with paper Triage Tags, color-coded in black, red, yellow and green, to signify one's degree of injury. These tags include space for writing pertinent medical information such as blood pressure, heart rate, and/or blood oxygen level at a point in time which serves as the primary means of field care documentation, and communication and information transfer between the field and the hospital. Similar to the TCCC in medical scenarios, noted limitations of current medical tags for civilian use include: 1) Limited space for recording medical data; 2) A format that allows only unidirectional changes in patient condition (worsening); 3) Tags that are not weather resistant, and are easily marred or destroyed; 4) A static and disconnected information repository, when real-time physiological data and/or patient information regarding victims and their status is critical to the continuity of field care management. The data tags allow for the creation of a patient centric local area network (patient centric network) for incorporating multiple data flows into one patient's EHR.

Physiological monitoring equipment such as ECG/EKG, pulse oximetry, heart rate monitors, temperature measurement equipment, and blood pressure measurement equipment is commonly used in healthcare facilities where they can be connected to a patient's EHR via the facilities network. Additionally, these physiological monitors can be made somewhat portable for medics and EMT's to bring to the patient for monitoring. These portable monitors are typically large reusable devices which must be shared among patients in the event of a mass casualty event. Patch style physiological monitors are available to be worn by patients and record physiological data. These patch style monitors are intended to communicate with proprietary data bridge devices that are not mobile and affixed in hospitals. These types of devices are typically not intended to communicate with an individual, patient-specific, mobile electronic medical record tag for display of patient status and data storage for transport with the patient and for later upload into the care facilities electronic health record. Additionally, the ability for the patient centric network to move with the patient allows for patient care alerts to be generated locally and shared with nearby or remote caregivers.

With the creation of a patient centric network of monitors, recording devices, and display devices the ability of providing clinicians and/or caregivers with real time patient condition and providing automated patient condition change or status alerts is possible. The patient centric network is particularly useful in conditions where limited or no network connectivity is available. In these cases the capture and local storage of patient physiological data incorporated into a local AI-driven decision support system provides a patient status alert system.

There is a need to streamline the documentation and communication of medical treatment performed early in emergency care situations, to continuously capture treatment or condition data in real time with accurate time stamps, and to communicate that information to the team of clinicians in a timely and effective manner. There exists a need for a low cost, mobile, patient specific, integrated physiological monitoring system that continuously records patient physiological data, stores such data in a patient specific data repository for later upload into the patient's EHR upon receipt at a care facility. There is also a need to communicate single or multiple patient's information to the caregiver or care team where network connectivity is limited or non-existent.

### SUMMARY OF THE DISCLOSURE

This disclosure provides an individual, cloud-based and on-device, patient monitoring system where a patient specific physiological monitoring device communicates with an electronic medical records tag to capture and report patient data to a mobile computer that is used by the caregiver. The mobile computer provides the caregiver with a hands-free solution to improve the interface between clinician providers and the EHR. The described systems and methods include the following: 1) overall system architecture, including the hardware and software components and interface, 2) software framework depicted in block diagrams, and 3) machine learning algorithms to enable the system to function. The described systems and methods can include the following core features: Continuous physiological monitoring of the patient's vital signs; flexible patient and caregiver health data entry methods allowing manual data entry, voice-driven data entry via automatic speech recognition, vision-based data entry via automatic object detection and classification, structured list or check box based data entry, and flexible context aware data entry; a machine learning algorithm, running in the cloud or on-device, that combines and analyzes human clinical data to compare data inputs with baseline data for establishing pertinent patient information (changes to a patient's physiological and neurological status, exposure to hazardous agents, environmental exposures, and risk assessments), and providing machine learning model output data and a clinical risk score; analysis of a casualty's dynamic vital sign data and cross-sectional EHR data (including, but not limited to radiographs, static vital sign data (i.e., first or last measurement sampled at a single point in time), and patient demographics data) to identify patients whose condition is worsening; alerting the caregiver to the worsening condition of the patient; robust functioning in acute/uncontrolled/non-traditional environments such as emergency departments (ED) or battlefield care situations; the ability to provide clinical decision support and EHR continuity across disconnected groups of care providers where different EHR systems are used to document the care of the same patient, such as when a patient is transferred from the emergency department of one hospital to another, or being transferred from a field aid station to a military hospital away from the front lines.

A method for treating a patient is provided, comprising the steps of obtaining cross-sectional data related to a patient, capturing time-series physiological data from the patient, inputting the cross-sectional data and the time-series physiological data into a trained machine learning model, and outputting a patient score from the machine learning model that provides an assessment of the patient's health.

In some embodiments, the patient score comprises an infectious disease diagnosis.

In one embodiment, the patient score comprises an indication of chemical-biological (CB) exposure.

In some examples, the patient score comprises a mortality assessment.

In one embodiment, the machine learning models for infectious disease diagnosis, CB exposure detection, and mortality risk prediction due to CB exposure use a RNN voting ensemble of sequential models. (or RNN voting ensemble models)

In some examples, clinical data entry modes include manual data entry, automatic/passive clinical data capture from wearable sensors, voice-driven automatic speech recognition, and automatic object detection from image and video data.

In one embodiment, the cross-sectional data is obtained from the patient's electronic health record (EHR).

In another embodiment, the cross-sectional data comprises an assessment of the patient from a medical provider.

In some examples, the cross-sectional data comprises patient medical history.

In one embodiment, the time-series physiological data is captured in real-time by sensors worn by the patient.

In some examples, the time-series physiological data is selected from the group consisting of activity, activity-based energy expenditure (AEE), accelerometry-based total daily energy expenditure (TDEE), arterial oxygen saturation (SaO2), arteriovenous oxygen difference (a-vO2), blood glucose level, cardiac waveform data, capnography (CO2 concentration), core body temp temperature (CBTemp), electrocardiogram (ECG or EKG), electrodermal activity (EDA), electroencephalograms (EEG), end-tidal CO2, extremity temperature, galvanic skin response (GSR) sensor for measuring skins electrical properties (conductance, resistance, impedance, capacitance), heart rate (HR), heart rate variability (HRV), hydration levels, Nerve agent time series data (ECG measures), motion, peripheral oxygen saturation (SpO2), Pulse Oximetry, photoplethysmogram (PPG), plethysmography, respiration rate (Resp or RR), skin temperature (Skin Temp), systolic, mean, and/or diastolic blood pressure (BP), spirometry data for pre- and post-particulate exposure, and time-series data for language classification.

In one embodiment, the method further comprises storing the time-series physiological data and the cross-sectional data on an electronic device worn by the patient.

In some examples, the trained machine learning model is developed and stored on an electronic device worn by the patient.

In one embodiment, the trained machine learning model is developed and stored on a cloud computing server.

A system configured to provide medical treatment to a patient is provided, comprising a personal computing device configured to record patient information and prior treatment information, a sensor unit configured to be worn by the patient and to record patient physiological measurements, an electronic data tag configured to store the patient physiological measurements, the patient information, and the prior treatment information, and a trained machine learning model configured provide a patient score that provides an assessment of the patient's health based on the patient physiological measurements, the patient information, and the prior treatment information.

In some embodiments, the personal computing device comprises a head-mounted display (HMD).

In other examples, the personal computing device comprises a smartphone.

In one embodiment, the sensor unit comprises a fabric sleeve with integrated sensors.

In some embodiments, the electronic data tag and sensor unit are configured to communicate by a wireless connection.

In one example, the personal computing device is configured to record patient information with a verbal input from a caregiver.

In some embodiments, the patient score is displayed on the electronic data tag.

In another embodiment, the patient score is displayed on the personal computing device.

A non-transitory computing device readable medium having instructions stored thereon is provided for determining a patient score that provides an assessment of the patient's health, wherein the instructions are executable by a processor to cause a computing device to: obtain cross-sectional data related to a patient; capture time-series physiological data from the patient; input the cross-sectional data and the time-series physiological data into a trained machine learning model; and output a patient score from the machine learning model that provides an assessment of the patient's health.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A-1B are examples of mobile patient monitoring systems for use in a hospital setting. This system includes a wireless patient monitor for communication with a proprietary data bridge for data upload for cloud processing.

FIG. 2 is a patient monitoring system for use in an in-hospital setting.

FIG. 3 is an example of a head mounted display (HMD) which incorporates a variety of sensors, data input methods, data display methods, and networking to record data.

FIGS. 4A-4B illustrate one embodiment of a HMD.

FIG. 5 is an example an electronic TCCC tag (E-TC3) or data tag.

FIG. 6 illustrates a physiological monitor sensor unit PMSU on a patient

FIG. 7A is an example of a patient centric network for communication between the datatag, PMSU, and HMD,

FIG. 7B illustrates a representative HMD screen with patient information displayed

FIG. 7C illustrates a representative data tag screen with patient information displayed

FIG. 7D is an example of the PMSU and data tag on a patient for transport

FIG. 8-11 illustrate components of the PMSU

FIG. 12 is a schematic of the components incorporated into a PMSU.

FIG. 13 is an alternate embodiment of the PMSU

FIG. 14 is an example of the on-device processing of patient data for providing patient data

FIG. 15 is an example of processing patient data in an on-device and off-device scenario for providing patient alerts.

FIG. 16 is a detailed illustration of the AI/ML model trained online and ported to a patient's device for running the model in an offline scenario.

FIG. 17 is an AI-driven system to detect clinical anomalies and provide Real Time medical alerts.

FIG. 18 is an example of a clinical decision support system running in offline mode, processing patient data offline, and uploading patient data to a cloud server once the patient reaches a care facility with network connectivity post use.

FIG. 19 is a clinical decision support system running online with continuous network connectivity.

FIG. 20-23 are schematic of the AI cloud server processing patient data (both time series data and cross-sectional data) for use by the patient's device and for use by clinicians and/or an electronic health record platform.

FIG. 24-25 are schematics of the use of automatic speech recognition (ASR) for entry and processing of patient data into the patient centric network.

FIG. 26-27 are schematics of the use of automatic object detection (AOD) for entry and processing of patient data into the patient centric network.

FIG. 28 is an example of the use of a trained AOD model on a user's HMD or mobile phone or wearable device for data entry into a patient centric network

FIG. 29 is a schematic of the system's machine learning algorithm for generating health risk predictions that uses a recurrent neural network (RNN) voting ensemble model with “ADD” aggregation that inputs cross-section and time-series data, consisting of static and dynamic features.

FIG. 30 is a schematic of aggregating predictions among voting models using “ADD” aggregation.

FIG. 31 is a schematic of aggregating predictions among voting models using “OR” aggregation.

FIG. 32 is a schematic of a sequential RNN model for chemical and biological exposure prediction, based on time-series data.

FIG. 33 is a schematic representation of converting machine learning model output data for CB detection and mortality risk prediction to a 2D visualization display for an Android smartphone application.

FIG. 34 illustrates the relationship between ML model outputs (for high and low mortality risk, shown as function of % mortality risk prediction as a function of days) and a 2D visualization display using a smartphone to view the ML model outputs.

### DETAILED DESCRIPTION

It is to be further understood that the present disclosure is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present disclosure. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Features, which are described in the context of separate embodiments, may also be provided in combination in a single embodiment. Conversely, various features, which are, for brevity, described in the context of a single embodiment, may also be provided separately or in any suitable sub-combination. The Applicants hereby give notice that new Claims may be formulated to such features and/or combinations of such features during the prosecution of the present Application or of any further Application derived there from.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

A head mounted display (HMD) may refer to one or more apparatus and/or one or more systems that are capable of accepting input from the user via a variety of input methods. Touch, voice, head tilt/motion, eye tracking are all examples of input methods into HMD systems. A head mounted display integrates visual display of images and text to the user, a microprocessor capable of executing instructions via software programs also known as apps or app. A head mounted display may also include computer memory, a digital camera, a motion sensor, and communicate with networks via wireless communication protocols.

A physiological monitoring sensor unit (PMSU) may refer to one or more apparatus and/or one or more systems that are capable of sensing and recording physiological data from the patient. Patient vital signs is a collection of physiological data required for diagnosis and treatment of an injury. Such data may include: electrocardiogram (ECG/EKG), photoplethysmogram (PPG), heart rate (HR), heart rate variability (HRV), core body temp temperature (CBTemp), Skin Temperature, (Skin Temp), systolic, mean, and/or diastolic blood pressure (BP), peripheral oxygen saturation (SpO2), arterial oxygen saturation (SaO2), Arteriovenous oxygen difference (a-vO2), respiration rate (Resp), motion, activity, blood glucose level, end-tidal CO2, galvanic skin response (GSR) sensor for measuring skins electrical properties (conductance, resistance, impedance, capacitance), hydration sensor, or other patient data needed to treat or diagnose a patient's condition.

“Software” may refer to prescribed rules to operate a computer. Examples of software may include: code segments in one or more computer-readable languages; graphical and or/textual instructions; applets; pre-compiled code; interpreted code; compiled code; and computer programs.

A “computer-readable medium” may refer to any storage device used for storing data accessible by a computer. Examples of a computer-readable medium may include: a magnetic hard disk; a floppy disk; an optical disk, such as a CD-ROM and a DVD; a magnetic tape; a flash memory; a memory chip; and/or other types of media that can store machine-readable instructions thereon. Non-volatile storage is a type of computer readable medium which does not loose the information stored inside when power is removed from the storage medium.

A “computer system” may refer to a system having one or more computers, where each computer may include computer-readable medium embodying software to operate the computer or one or more of its components. Examples of a computer system may include: a distributed computer system for processing information via computer systems linked by a network; two or more computer systems connected together via a network for transmitting and/or receiving information between the computer systems; a computer system including two or more processors within a single computer; and one or more apparatuses and/or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those made through telephone or other communication links. A network may further include hard-wired connections (e.g., coaxial cable, twisted pair, optical fiber, waveguides, etc.) and/or wireless connections (e.g., radio frequency waveforms, free-space optical waveforms, acoustic waveforms, etc.). Examples of a network may include: an internet, such as the Internet; an intranet; a local area network (LAN); a wide area network (WAN); and a combination of networks, such as an internet and an intranet.

Exemplary networks may operate with any of several protocols, such as Internet protocol (IP), asynchronous transfer mode (ATM), and/or synchronous optical network (SONET), user datagram protocol (UDP), IEEE 802.x. Bluetooth is an example of an IEEE standard under IEEE 802.15.1. WIFI is an example of an IEEE standard under IEEE 802.11x. WIFI may be implemented as a traditional network or as a peer to peer (P2P) network architecture where two electronic devices communicate directly without an intermediary device. WIFI direct and Bluetooth are both examples of a peer to peer (P2P) network.

Embodiments of the present disclosure may include apparatuses for performing the operations disclosed herein. An apparatus may be specially constructed for the desired purposes, or it may comprise a general-purpose device selectively activated or reconfigured by a program stored in the device.

Embodiments of the disclosure may also be implemented in one or a combination of hardware, firmware, and software. They may also be implemented as instructions stored on a machine-readable medium, which may be read and executed by a computing platform to perform the operations described herein.

The term user, operator, physician, nurse, EMT, medic, or clinician refers to the person delivering care to a patient. The term patient, casualty, accident victim, or injured refers to the patient receiving care.

The term patient data refers to any background information, physiological data, or patient history data that may be useful in classifying, diagnosing and/or treating a patient's medical condition. Background information may be: gender, age, race, condition history, patient medication, allergies, pale or ashen skin color, bluish or gray tinge to lips or fingernails, nausea or vomiting, enlarged pupils, weakness, fatigue, dizziness, fainting, change in mental status or behavior, anxiousness or agitation, or other background information. Physiological data may be metrics such as; heart rate, heart rate variability, vo2max, Plethysmography, SpO2, Pulse Oximetry, respiration rate, Capnography (CO2 concentration), cardiac waveform data, electrocardiogram, (ECG or EKG), brain function waveform data, Electroencephalograms (EEG), core body temperature, extremity temperature, Electrodermal activity (EDA), Blood Pressure (BP), patient shock, and other physiological metrics. Examples of patient history may be injury type, injury location, injury time, medications administered, medication dosage and time of administration, medication interactions, tourniquet administration, blood loss, ATMIST data, MARCH data, PAWS data, or other patient history or treatment data.

Wearable sensing technology offers the unique opportunity to implement a nonintrusive monitoring system, and a tool for observing and analyzing the complex human-machine-environment system engaged in specific tasks. The ability to predict soldier training limits and work-rest cycles previously relied on generalized models based on estimated inputs about individuals and ambient conditions. Wearable physiological monitoring can now provide predictions about an patient's health and/or performance from an individual's real-time physiological state.

Examples of patient or soldier performance and readiness applications include the assessment of thermal-work strain limits, alertness and fitness for duty, impending musculoskeletal injury, and physical fatigue limits. Health/medical management applications include: casualty detection, remote triage, and medical management; chemical/biological threat agent exposure for early detection and management; environmental/military occupational exposure dosimetry; health readiness behavioral management tools; neuropsychological status (mood and cognitive status); pulmonary exposures limiting performance; and specialized environmental exposures (e.g., hypoxia, peripheral cold monitoring).

Sensors are a crucial aspect of performance monitoring systems, which have gained in sophistication while decreasing in size and cost. Dominant sensors in today's market fall into three categories: physiological, kinetic, and agent detection. When working with smart devices and body sensors, one must consider requirements such as comfort, flexibility and washability. At the same time, many wearable systems are meant to be worn during rugged activity. Soldiers in the field need wearable smart systems that can withstand a wide range of temperatures. The materials need to provide effective shock and vibration resistance, as well as resistance to chemicals or solvents that might otherwise destroy a commercial device. Interconnections and electronics must be unobtrusive and durable. This requires reliable terminations that are insulated, robust and waterproof; flexible, with antenna and transceiver solutions; small, dryable batteries; and crimp resistant printed circuit boards/flexible printed circuits (PCB/FPCs).

Developing a performance monitoring system requires designing a platform with algorithms that can incorporate many of the disparate sensors that are currently available. Current commercial systems generally do not satisfy the requirements for use, since raw data from such devices often cannot be easily accessed or combined to provide meaningful decision support. Even when systems provide more than raw physiological data, computed information is usually based on closed-system architectures that cannot be properly reviewed and validated, making the output unusable. For military or first responder cases, unsecure and power-demanding connections and proprietary architectures cannot be easily integrated into tactically secure systems and communications networks. Likewise, for military applications, systems should not add significant weight to the soldier, nor require daily recharge or battery replacement. Thus, reduced size, weight and power (SWaP) is critical to acceptability and tactical usability.

### Example Datasets and Corresponding AI/ML Models

For far forward field care where injuries are sustained from military munitions, time-series, real-time data capture metrics can be AI models including those to predict hemodynamic instability based on acute changes in vital sign data, compared to baseline measurements; AI models to predict long-term endurance, fatigue, and physiological performance, based on RT accelerometry data, temperature, HR, hydration measurements, etc. Both require access to time-series physiological data captured from multiple patients over a sustained period of time (baseline+variable). Cross-sectional data can include wound assessment—based on data points (stage, status, location) to build a classifier for wound healing; Traumatic brain injury (TBI) assessment based on neurological measures/data points to build a classifier for traumatic brain injury (TBI); Spinal cord injury assessment based on neurological and physical measures to build a classifier for spinal cord injury in combat.

For enduring threats of disease from the use of chemical, biological, radiological, and nuclear (CBRN) weapons during Multi-Domain Operations (MDO): Time-series data (example datasets needed for AI development): RT physiological/vital sign data for 2-3 measures (i.e. HR and Temp) for: Patients exposed to a radiological/biological/chemical/nuclear agent vs. Baseline data for un exposed patients (captured from same devices). Spirometry data for exposure to particulate, pre- and post-deployment comparative datasets to show how lungs are affected by deployment. Nerve agent time series data (ECG measures), captured over 24 hours from multiple patients.

Cross-sectional data is a type of data collected by observing subjects at the one point or period of time. Cross-sectional data may also consist of one or more of the following: demographics data, height, weight, race, sex, radiographic data, X-ray, CT Scan, MM, wound images, static vital sign data consisting of a first and last measurement (e.g. measurements captured at a single time point), gender, age, condition history, patient medication, allergies, pale or ashen skin color, bluish or gray tinge to lips or fingernails, nausea or vomiting, enlarged pupils, weakness, fatigue, dizziness, fainting, change in mental status or behavior, mood and cognitive status, anxiousness or agitation, visual signs of shock, or other healthcare and patient background information. Examples of patient history may be injury type, injury location, injury time, medications administered, medication dosage and time of administration, medication interactions, tourniquet administration, blood loss, ATMIST data, MARCH data, PAWS data, or other patient history or treatment data.

Cross-sectional data may include image data and frames from video data captured at a single point in time.

Additional examples of cross-sectional data include toxidromes, or a pattern of symptoms and signs (syndrome) due to exposure to a toxic substance. Notable toxidromes include nerve agent intoxication and opioid overdose, and visual symptoms of human exposure to mustard gas, medicines, poisons, or other hazardous agents; ChemDX cholinesterase detection data, and the CHEM-IST database (based on toxidromes).

Time series data, also referred to as time-stamped data, is a sequence of data points indexed in time order. Time-stamped is data collected at different points in time. These data points typically consist of successive measurements made from the same source over a time interval and are used to track change over time.

Examples of time-series data include, but are not limited to the following: Activity, activity-based energy expenditure (AEE), accelerometry-based total daily energy expenditure (TDEE), arterial oxygen saturation (SaO2), Arteriovenous oxygen difference (a-vO2), blood glucose level, cardiac waveform data, Capnography (CO2 concentration), core body temp temperature (CBTemp), electrocardiogram (ECG or EKG), Electrodermal activity (EDA), Electroencephalograms (EEG), end-tidal CO2, extremity temperature, galvanic skin response (GSR) sensor for measuring skins electrical properties (conductance, resistance, impedance, capacitance), heart rate (HR), heart rate variability (HRV), hydration levels, Nerve agent time series data (ECG measures), motion, peripheral oxygen saturation (SpO2), Pulse Oximetry, photoplethysmogram (PPG), Plethysmography, respiration rate (Resp or RR), Skin Temperature (Skin Temp), systolic, mean, and/or diastolic blood pressure (BP), Spirometry data for pre- and post-particulate exposure, Time-series data for language classification.

The disclosed AI-driven clinical decision support tool that combines static and dynamic clinical data to detect CBRN threats and exposures, and to predict changes to a casualty's heath status due to a CBRN exposure, could involve a range of potential exposures. Examples of CBRN agents include: chemical agents such as nerve agents, cholinesterase inhibitors, blistering agents, cyanides, physical and mental incapacitants, toxic industrial chemicals (TICs), riot-controlled agents (RCAs), ethylene oxide, formaldehyde, glutaraldehyde; pharmaceuticals and drugs such as cancer chemotherapy, antiviral treatments, hormone regimens; waste anesthetic gases such as halogenated anesthetics (e.g., halothane, enflurane, isoflurane, and desflurane); biological agents and infectious diseases such as live agents (bacteria, viruses, and fungi), Anthrax, Avian flu, bloodborne pathogens, Cytomegalovirus (CMV), COVID-19, Ebola, Measles, Methicillin-resistant Staphylococcus Aureus (MERS), Norovirus, Pandemic influenza, Severe Acute Respiratory Syndrome (SARS), Tuberculosis, and the Zika virus; toxins derived from bacteria, fungi, plants and animals (i.e., venom), radiological material such as alpha, beta, and gamma particles, and neutrons; nuclear material; lung particulate exposures as seen by spirometry pulmonary function test data; and heat exposures as evidenced by elevated body temperature, slurred speech, abnormal thinking behaviors, heavy sweating, hot, dry skin, headache or nausea, thirst/dehydration, and decreased urine output.

Ai/ML Models:

The term Artificial intelligence (AI) is a field of computer science which makes a computer system that can mimic human intelligence. It is comprised of two words “Artificial” and “intelligence”, which means “a human-made thinking power.”

Deep learning imitates the human brain's neural pathways in processing data, using it for decision-making, detecting objects, recognizing speech, and translating languages. It learns without human supervision or intervention, pulling from unstructured and unlabeled data. Deep learning processes machine learning by using a hierarchical level of artificial neural networks, built like the human brain, with neuron nodes connecting in a web. While traditional machine learning programs work with data analysis linearly, deep learning's hierarchical function lets machines process data using a nonlinear approach.

Machine learning (ML) enables a computer system to make predictions or take some decisions using historical data without being explicitly programmed. Machine learning uses amassive amount of structured and semi-structured data so that a machine learning model can generate accurate result or give predictions based on that data.

Machine learning works using algorithms that learn on their own using historical data. ML models work only for specific domains. Machine learning is being used in various places such as for an online recommender system, for Google search algorithms, Email spam filter, Facebook Auto friend tagging suggestion, etc. It can be divided into three types: Supervised learning, Reinforcement learning, Unsupervised learning.

Supervised machine learning algorithms can apply what has been learned in the past to new data using labeled examples to predict future events. Starting from the analysis of a known training dataset, the learning algorithm produces an inferred function to make predictions about the output values. The system can provide targets for any new input after sufficient training. The learning algorithm can also compare its output with the correct, intended output and find errors to modify the model accordingly.

In contrast, unsupervised machine learning algorithms are used when the information used to train is neither classified nor labeled. Unsupervised learning studies how systems can infer a function to describe a hidden structure from unlabeled data. The system does not figure out the right output, but instead explores the data and can draw inferences from datasets to describe hidden structures from unlabeled data.

Semi-supervised machine learning algorithms fall somewhere in between supervised and unsupervised learning, since they use both labeled and unlabeled data for training—typically a small amount of labeled data and a large amount of unlabeled data. The systems that use this method can considerably improve learning accuracy. Usually, semi-supervised learning is chosen when the acquired labeled data requires skilled and relevant resources to train it/learn from it. Otherwise, acquiring unlabeled data generally does not require additional resources.

Reinforcement machine learning algorithms is a learning method that interacts with its environment by producing actions and discovers errors or rewards. Trial and error search and delayed reward are the most relevant characteristics of reinforcement learning. This method allows machines and software agents to automatically determine the ideal behavior within a specific context to maximize its performance. Simple reward feedback is required for the agent to learn which action is best; this is known as the reinforcement signal.

Artificial intelligence Machine Learning model may be singular or may employ multi-modal processing where multiple data sources (time-series and cross-sectional) are combined to provide a more accurate model of actual patient status. For example, heart rate, blood pressure, and temperature time series measurements may be combined in an AI/ML model to determine the cardiovascular condition of a patient and predict the patient's status in the future. Other examples of multi-modal vital sign analysis are: to predict health outcomes for critically ill patients based on heart rate, arterial blood pressure, and respiratory rate; to compare electronic recordings of hemodynamic and electrocardiographic waveforms of stable and unstable patients in critical care units, operating rooms, and cardiac catheterization laboratories; and to predict health outcomes of patients diagnosed with TBI based on multi-channel recordings of ECG, arterial blood pressure (ABP), and intracranial pressure (ICP).

Algorithms for Data Analysis and Medical Alerts

Based on recent advances in multimodal machine learning, this disclosure provides systems and methods that can include an algorithmic framework that can combine raw vital sign data with different signal-to-noise (SNR) characteristics acquired from wearable sensors to indicate a patient's health status (e.g., if he/she is hemodynamically unstable or actively crashing). In particular, the systems described herein can use multimodal diffusion map analysis and anomaly detection based on multimodal deep learning to design algorithms that provide quantitative measures for a range of acute health conditions. These measures come with an associated confidence score. Moreover, these algorithms are designed to be computationally- and memory-efficient and thus to run either on- or off-device. Multimodal machine learning allows for the generation of predictive analytics to estimate a patient's future condition based on the trajectory of current data. Raw vital sign data represents a special case of multimodal data. The analysis of multimodal data poses numerous challenges due to the fact that the data are generated by potentially different processes, due to the inherent heterogeneity of the data, and the possibly different signal-to-noise ratios of each data modality. Existing techniques to indicate if a patient is experiencing cardiopulmonary arrest, shock, and/or hypothermia based on raw vital sign data, are not adequate for these purposes. For instance, the Modified Early Warning Score and Cardiac Arrest Risk Triage are not accurate enough for a military and/or high-trauma environment in which our system will be deployed. Furthermore, these scores use basic statistical methods that cannot take into account the diversity of the SNR in the signal sources nor any advanced characteristics that may be present in the vital sign signals. Moreover, these scores are tailored to cardiac arrest, but are not able to predict other medical emergencies.

This disclosure provides a framework for an AI-driven clinical decision support tool that is designed to support two data input types—time series data and cross-sectional data.

With time-series data, the clinical decision support (CDSS) tool provided herein can predict the next values (i.e. HR, SpO2) based on the previous values captured, due to the dataset upon which the model was trained. Cross-section data inputs are used to develop classifiers of health conditions or disease states, based on a range of data points per patient that are not time-dependent.

Time-series data can also be used for classification. In the case of language classification, if the system herein is presented with a number of audio recordings, and the language spoken on each audio file is labeled, the machine learning algorithm can be configured to predict the language when presented with a new recording.

Learning from both time-series and cross-sectional data can be framed as supervised learning problems. Time-series data requires the extra steps of conversion by using sliding windows and extracting features. Windowing is the process of training the model on a small range of dates and then testing on a range of dates immediately following.

The systems and methods provided herein can be configured for both offline and online use: Offline use is when the system/methods/algorithms run from or are implemented locally on a phone or wearable device. Online use is when the system/methods/algorithms run from or are implemented a cloud or remote server location.

Real-time & Non-real time (asynchronous): The clinical decision support system herein provides real-time analytics; or data can be captured and stored for future analysis.

Manual Data Entry/Capture & Automatic data capture: Manual data entry includes clinical information typed (by a clinician or medic) into a patient's EHR or manually entered into a phone or computer from reading the vital signs from a patient's vital sign monitor.

Automatic passive/active data capture includes automatic inputs such as data from vital sign monitors, images, speech and other audio/video data from a phone or wearable device.

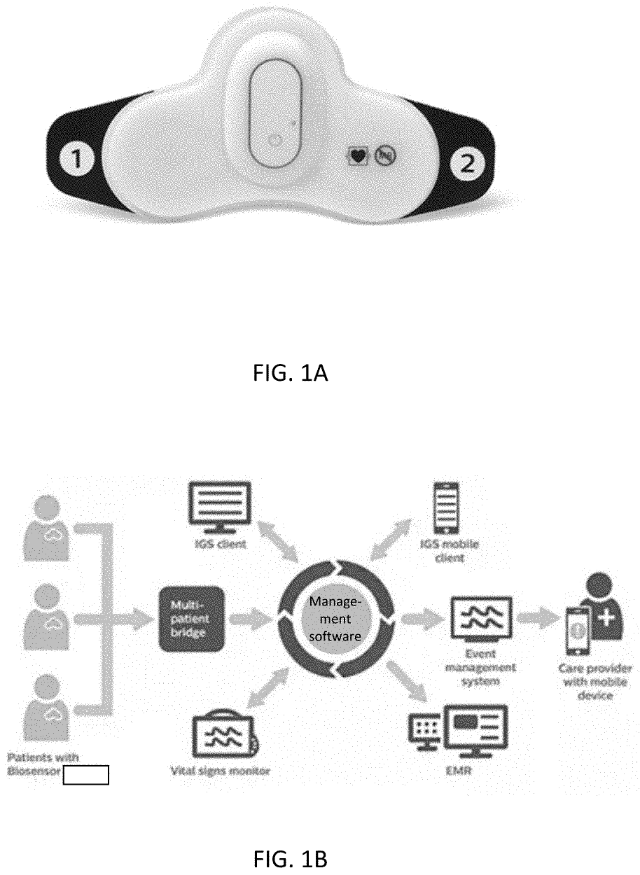

FIG. 1A is one example of a commercially available patient monitoring device for use in-hospital for acquiring patient data. This device is a patch style patient monitor with Bluetooth connectivity to in-hospital patient monitoring systems. The system communicates with a proprietary data bridge shown in FIG. 1B for data collection. These data bridge type devices typically have limited range and are not configured for mobile use.

FIG. 1B is one example of a commercially available software system for use in an in-hospital setting. This system communicates with the patch of FIG. 1A to monitor a patient and provide on-line patient status alerts. The data bridge communicates with multiple wearable patch sensors to provide data information for processing and distribution.

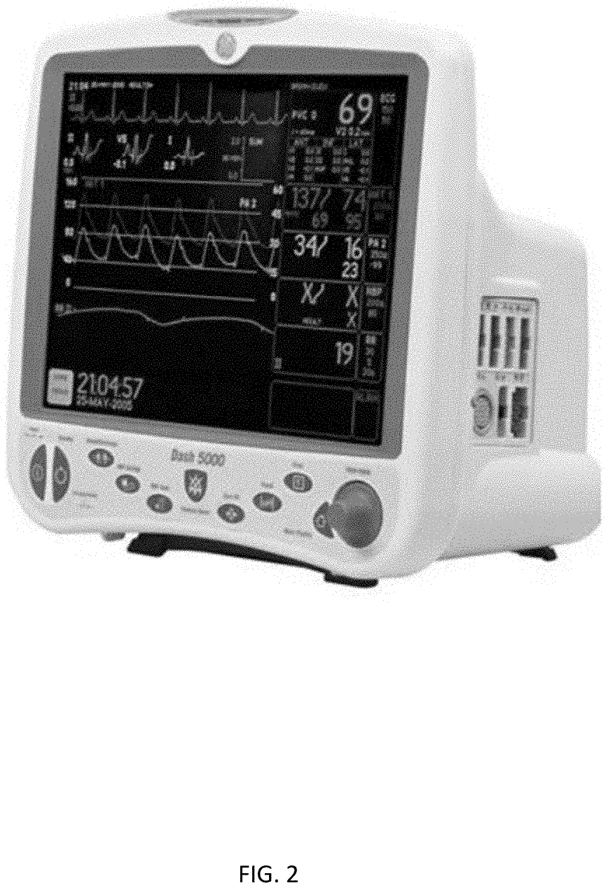

FIG. 2 is a another commercially available patient monitor for monitoring vital signs of a patient in a hospital.

Head Mounted Display for Caregivers

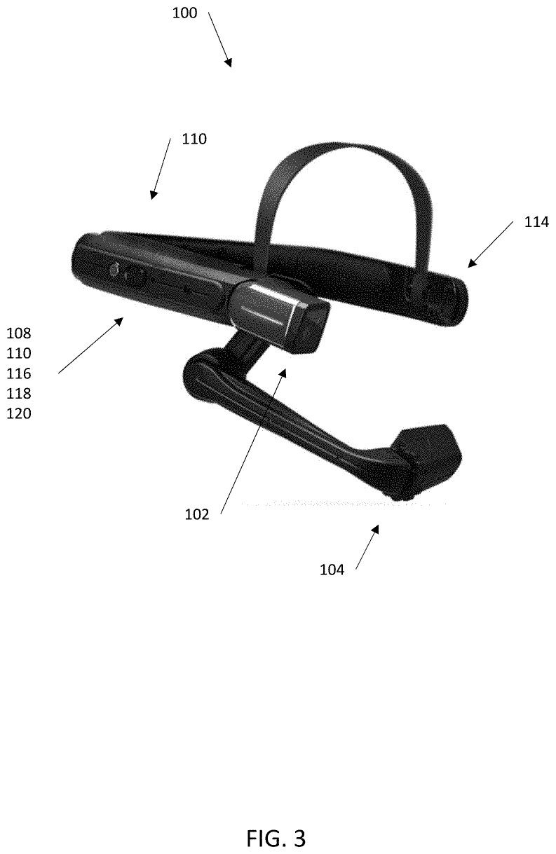

FIG. 3 is an example of a head mounted display (HMD) 100 which incorporates a variety of sensors, data input methods, data display methods, and networking to record data. For example, the HMD can include a camera 102, display 104, and a frame 108 that houses or supports a processor 110, communications chip 112 (such as Bluetooth or wife), batteries 114, a trackpad 116, control buttons 118, and sensors 120 (such as accelerometers, gyroscopes, magnetometers, altitude sensors, humidity sensors, etc.).

This head mounted display is capable of receiving input through a microphone and responds to voice commands. The microphone is configured to incorporate noise-cancelling techniques to provide a noise reduced voice signal to the voice to text processor in the HMD and additional hardware. This microphone can be configured to be of a boom style, and/or may be configured to be noise cancelling, where an ambient microphone records the ambient noise and outputs an inverted noise signal into the boom microphone, reducing the perceived loudness of the noise while boosting the clarity of the voice signal. To improve the performance of the speech recognizer, a boom microphone may be implemented. If the distance from a user's mouth to the HMD's built-in microphone is 100 mm, a microphone mounted on a boom that extends to the front of the speaker's mouth will reduce the distance to 10 mm. Because the sound intensity from a point source of sound will obey the inverse square law if there are no reflections or reverberation, the intensity of the speech signal will theoretically be 20 dB higher, leading to a considerable improvement in signal-to-noise ratio (SNR).

The HMD also configured to be controlled via touch/trackpad/button commands. The HMD is capable of: performing on-board processing of data from the voice commands, displaying a menu-based treatment checklist, broadcasting audio output, and transmitting patient data via network protocols. The accelerometer, gyroscope, magnetometer, altitude sensor, and humidity sensors are able to record data relating to patient treatment. Additionally, the HMD is configured to provide a digital clock or chronometer to record the time of treatment. The HMD is also configured to include an auto-focus camera for recording photographic and video images of the patient during treatment. The HMD incorporates a microprocessor with onboard RAM, flash non-volatile storage, and runs an operating system.

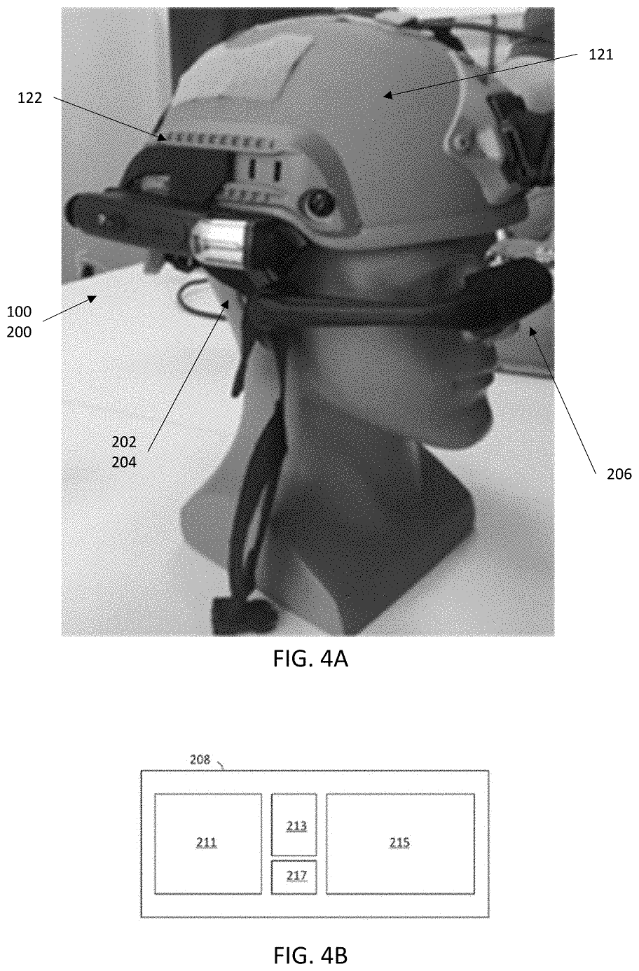

FIGS. 4A-4B illustrate one embodiment of an HMD 100, that can be mounted to a protective helmet 121 for providing protection to the caregiver. The HMD may be mounted to the helmet via accessory mounting rails 122 on the side of the helmet. As shown in FIG. 4B, the electronics compartment can house a processor 211, a non-transitory computer-readable storage medium 213 configured to store a set of instructions capable of being executed by the processor, and an energy source 215 such as a battery to power the device. The electronics compartment can also include additional electronics 217 which can be a microphone, wireless communications electronics such as WIFI, cellular, or Bluetooth chips that enable the assessment device to communicate with other devices and computers wirelessly, imaging processing microchips, gyroscopic position and orientation sensors, eye tracking sensors, eye blink sensors, touch sensitive sensors, speakers, vibratory haptic feedback transducers, stereoscopic cameras, or other similar electronics and hardware typically found on smartphones and digital devices. While the HMD 100 is illustrated as a hands-free, wearable device, in other embodiments the assessment device can be a smartphone, PC, tablet, or other electronic device that includes the components described above including a camera, a processor, non-transitory computer-readable storage medium, a display, and an energy source.

The processor 211 can be configured to control the operation of the assessment device, including executing instructions and/or computer code stored on the non-transitory computer-readable storage medium 213, processing data captured by the camera 202 and additional sensor(s) 204, and presenting information to the display 206 for display to the user of the device. In some embodiments, the processor is configured to determine the dimensions of a wound and to overlay a digital ruler or measurement scale on top of digital images of a wound for documentation purposes. In some embodiments, the processor can determine the dimensions of a wound without requiring a physical measurement device or reference marker to be positioned on or near the wound. The modified image with the overlaid digital ruler or measurement scale can be stored on the non-transitory computer-readable storage medium 213, displayed on the display 206, stored in the patient's electronic medical record, and/or transmitted to another computer or device for storage, display, or further manipulation or study.

The processor can further be configured to affix or overlay patient information such as name, date of birth, and other identifying information from the patient or the patient's chart onto the display. This information can be acquired automatically by the processor from an electronic medical tag, can be entered manually by the user, or can be verbally spoken into the microphone of the HMD and processed with speech recognition software. Additionally, the processor 211 may be configured to offload processor intensive operations to an additional computer, mobile phone, or tablet via the wireless connections such as WIFI, cellular, or Bluetooth; or transferred to a cloud-based platform and data repository.

The camera 202 can be configured to capture digital images and/or high-resolution video which can be processed by the processor 211 and stored by the non-transitory computer readable storage medium 213, or alternatively, can be transmitted to a separate device for storage such as the data tag described herein or a cloud-based data repository. The camera can include a zoom lens or a fixed focal length lens, and can include adjustable or auto-focus capabilities or have a fixed focus. In some embodiments, the camera can be controlled to take images/video by pressing a button, either on the HMD itself or on a separate device (such as a smartphone, PC, or tablet). In other embodiments, the user can use voice control to take images/video by speaking into the microphone of the HMD or separate device (such as a smartphone, PC, or tablet), which can process the command with speech recognition software to activate the camera. In one embodiment, the camera 202 may be a stereoscopic camera with more than one lens which can take simultaneous images of the patient at a known camera angle between the camera focusing on the same point of the image. The stereoscopic images along with the camera angle can be used to create a three-dimensional image of the patient.

The additional sensor(s) 204 can include an infra-red sensor, optical sensor, ultrasound sensor, acoustic sensor, a laser, a thermal sensor, gyroscopic position and orientation sensors, eye tracking sensors, eye blink sensors, touch sensitive sensors, speakers, vibratory haptic feedback transducers, stereoscopic cameras, or the like. The additional sensor(s) can be used to provide additional information to the processor for processing image data from the camera or for storing patient data or photographs/video onto the data tag.

The display 206 illustrated in FIG. 4A. is a monocle style display that allows a user to flip the display down for use and flip up when the display is not needed. The display may be opaque or may be see-through, for example, an OLED screen with multiple layers of glass or transparent material surrounding the OLED. While the HMD 100 of FIG. 4A includes a single display 106 in front of only one eye of the user, it should be understood that in other embodiments, the HMD can include two displays (one in front of each eye of the user) or a single large display that extends across the periphery of both eyes of the user.

The HMDs of described herein can be a version of a wearable computer, which is worn on the head and features a display in front of one or both eyes. The HMD is configured to provide a portable, hands-free environment. The environment of the HMD is configured to provide a user to computer interface. The preferred embodiment of the computer interface is a hands-free interface to allow caregivers to provide care with their hands while the computer interface displays information to the caregiver and/or the caregiver records patient data. Types of hands-free interfaces include voice-based, eye-based, electromyographic (EMG)-based, gesture-based, and electroencephalographic (EEG)-based.

The HMDs of described herein can be configured to have a voice-based user interface (VUI): Voice user interfaces are uniquely based on spoken language, learned implicitly at a young age, whereas other user interfaces depend on specific learned actions designed to accomplish a task, such as selecting an item from a drop-down menu or dragging and dropping icons. The performance of the VUI is naturally dependent on accurate speech-recognition software, described below.

The HMDs described herein may further be configured to incorporate an automatic speech recognition (ASR) system. The ASR on a mobile/wearable processor would run continuously, provide a low-latency response, have a large vocabulary, and operate with minimal battery drain. The system of FIG. 4A is further configured to incorporate Deep Neural Network(DNN) support in the ASR to improve speech recognition. The ASR of the system further is configured to have a to customized language model specific to medical, EMT, and/or military application environments.

The HMDs described herein are further configured to include software and hardware capable of reading patient information off of a patient wrist band or patient identification card. Bar-code scanning, optical character recognition (OCR), radio frequency identification (RFID), 2-d barcode, or other data entry methods may be employed. An example of OCR data entry is the automatic reading of a patient's name or other information off of a military identification tag.

An alternative embodiment of the HMD data input/output device would be a clinical data capture software application running on the caregiver's smartphone. The application would include all the functionality of the HMD device without being head mounted. The caregiver would enter clinical data via voice, touch, or button-based input, while reading patient information from the patient's data tag through a peer-to-peer network, a wide area network, and/or local area network.

Patient Records Tag

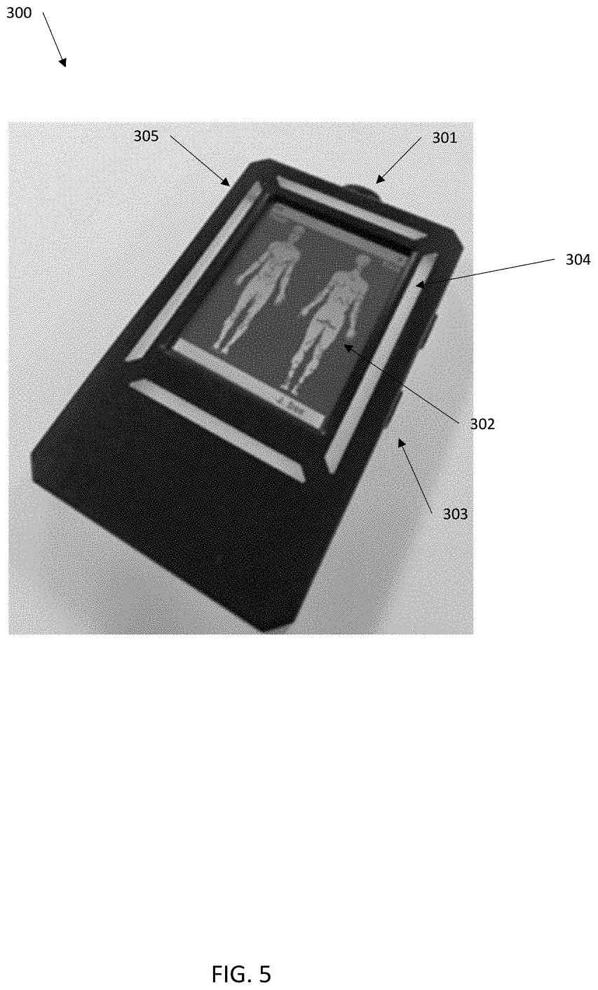

FIG. 5 is an electronic patient record tag (E-TC3) 300 or data tag, which is configured to be attached to the patient at the time of treatment by the caregiver. The tag may be affixed to the patient with: a lanyard, a strap, a wrist or ankle band, adhesive, an armband, tape, hook and loop fasteners, safety pins, buttons, snaps, or other methods. A hole for affixing a lanyard is shown in FIG. 5 (301).

The HMD is the interface between the user and the data tag. The data tag is configured to contain a data storage microchip, a microprocessor, and a battery. The data transmitted to the data tag from the HMD is stored in the data tag on internal non-volatile storage such as flash memory, hard drive, or other non-volatile memory methods. The data tag is further configured to contain a display 302, which will display selected patient information on the external surfaces of the data tag. The display is constructed as a liquid crystal display (LCD) display however LED, OLED, or e-ink style displays may be used. The display may be monochrome, or full color, or a combination of each, and is configured to display images, text, icons, or a combination thereof. The display may incorporate an array of LED lights 304 either integrated or separated from the main display to indicate patient status. The display may include a touch screen interface for scrolling or changing pages to display more patient information. The display of patient information is to inform clinicians, transportation EMTs or other care givers who are not wearing a HMD. The patient's vital signs, triage status, injury location, treatments given, drugs or other medications administered, time of drug administration, tourniquets applied, time of tourniquet application, and or time of next tourniquet change and/or loosening are selectively displayed so the caregivers have the critical patient information clearly and easily at hand. The tag may also display patient allergies, drug combination errors, and/or clinical decision support recommendations. The tag may contain physical buttons 303, dials, touch screen, or other user input methods to collect user input. The tag may also be equipped with a timer and a speaker to provide an audible alarm to alert caregivers of clinical care which is required at a certain time. For example, such an audible alarm would be useful to alert caregivers that a tourniquet needs to be adjusted within a certain period of time after tourniquet application. The tag may be configured to be a function of a smartwatch which is pre-worn by the patient. The data tag also includes an array of patient status alert lights (304), which can display patient status. These alert lights are full color and capable of flashing in patterns to communicate information. For example, patients who are of most critical condition will have a tag which displays red, while patients who are in serious condition may have a tag which displays yellow. The lights are also dimmable to adjust brightness for optimal viewing in ambient lighting conditions. The dimming level required is sensed via an ambient light sensor (305) integrated into the tag casing and exposed to the exterior. The patient status lights may be initiated by the caregiver through voice or menu commands with the HMD. Alternatively, the patient status lights may be initiated by the tag through patient vital sign monitoring algorithms which are running on the tag and monitoring the data output from the PMSU.

At the time of care, the tag and a PMSU (described below) can be affixed to the patient and turned on. The tag and PMSU will pair and will begin acquiring patient vital signs. This establishes a patient centric network (PCN) for coordination of data flows from various sensors to a central data tag. Vital signs will be displayed on the tag's display. The tag may be attached to the PMSU directly via hook and loop fasteners. The system of FIG. 5 depicts the PCN system monitoring a patient's condition when the patient is prepared for transport. This embodiment utilizes the wireless connection capability of the PCN system to allow the attachment of the data tag outside the patient's wrap blanket (not shown for clarity) for easy display of the patient's vital signs, triage status, condition, or hemodynamic score or CMDS score. The data tag may be affixed to the blanket via an adhesive sleeve, adhesive, strap, or other fixation method. The caregiver may then elect to pair their HMD or other mobile computer with the tag for wireless communication and monitoring of the patient. The PCN is continually recording patient metrics and treatment information while the patient is transported to the next level of care.

The data tag of FIG. 5 is configured to include a battery for powering the functionality described above. The battery is sized such that the data tag is powered for a prolonged period to allow for prolonged field care of patients during a casualty event. Typically, prolonged field care is less than 3 days. Alternatively, as battery power is critical to the function of the device, the tag of FIG. 5 can be configured to have a battery installed where the battery is not drained during storage. Upon affixing of the data tag to the patient a switch is flipped, or an insulating film is released from the battery contacts to permit the battery to power the data tag. The tag is then paired with the HMD via Bluetooth, WIFI direct, or other communication protocols, and data storage and display may commence. As treatment occurs, physiological and treatment data is recorded by the PMSU and the HMD and stored on the data tag. Alternatively, the tag may include a microphone and touchscreen interface to collect data from the treatment of the patient without the use of an HMD.

Once the patient is stable the patient is transported to a care facility such as a hospital, field aid station, or other fixed medical facility. At that facility, there is a reader configured to read the data off of the data tag and incorporate the patients' medical information stored on the tag into the hospital's electronic health record system (EHR). Once the data is read from the data tag, the data tag can be configured to destroy the data inside to protect patient privacy.

The data tag of FIG. 5 is configured to be disposed of after the patient is transferred to a care facility.

The visual displays of the HMDs or the tag described herein are configured to provide the user with an augmented reality computer environment where menu commands are displayed on the inside of the lenses of the glasses. The menu system can be configured to be activated by voice commands, touch, or button commands. The menu system is configured to provide a treatment checklist to the user for treatment of the patient. The treatment checklist is stepped through by the user who is administering care with both hands, while the HMD is providing treatment information to the user and recording patient information via voice commands by the user. The patient information is then transmitted to the tag of FIG. 5. Additionally, by monitoring the vital sign measures simultaneously, an assessment or score of a patient's hemodynamic stability and/or health status can be obtained or CMDS score. When the patient's score drops below a given threshold, the system will trigger an alert to caregivers that the patient needs immediate medical attention. This monitoring may be performed on the tag, on the HMD, on a smartphone or remote PC, or on multiple devices simultaneously. The tag may alert a caregiver to the patient status and that treatment or patient attention may be required.

An alternative embodiment of the medical records tag may be a software application residing on a smartphone. The smartphone application may display the patient condition pages as shown in FIG. 5 and be configured to be placed on the patient and travel with the patient to the point of care.

Physiological Monitor Sensor Unit (PMSU)

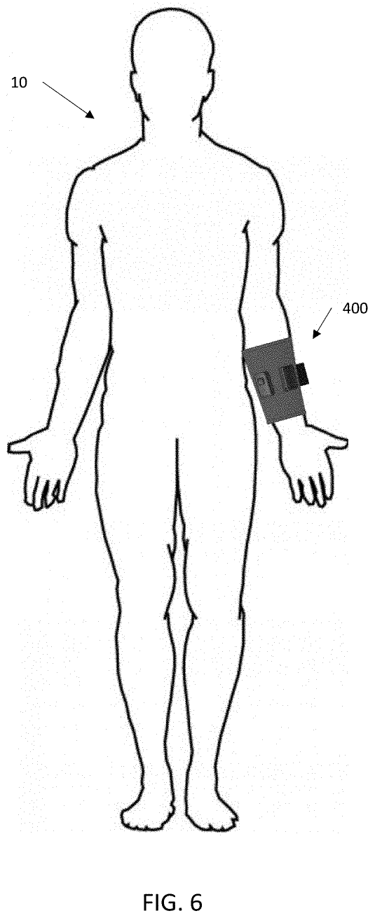

The system shown in FIG. 6 includes a Physiological Monitor Sensor Unit (PMSU) which is shown as an armband in this embodiment. The PMSU is placed on the patient's 10 wrist upon initiation of treatment.

Potential locations and configurations of sensors for human performance monitoring include, but are not limited to, a wrist/watch-like configuration; chest or trunk-based configuration for capturing data such as heart rate and accelerometry-based total daily energy expenditure (TDEE); boot-worn configurations for capturing foot-contact time to measure activity-based energy expenditure (AEE), to classify types of activity, and to track changes in aerobic fitness levels; arm-based systems to capture cardiac function through heart rate, pulse, and blood pressure measurements, and as markers for workplace fatigue; and ear-worn devices; among others.

The PMSU includes wearable, self-monitoring sensors to monitor and capture vital signs, such as ECG, SpO2, HR, Temp. The PMSU can additionally include electronics to enable additional functionality, including batteries, one or more CPU's or processors (for processing sensed signals from the patient), onboard memory for data storage, and wireless communications for transmitting sensed and stored parameters to other devices (such as a PC, smartphone, tablet, HMD, electronic tag, or other computing system).

The PMSU may additionally include wearable environmental sensors such as dosimeters to detect radiation levels and sensors to monitor personal particulate monitors.

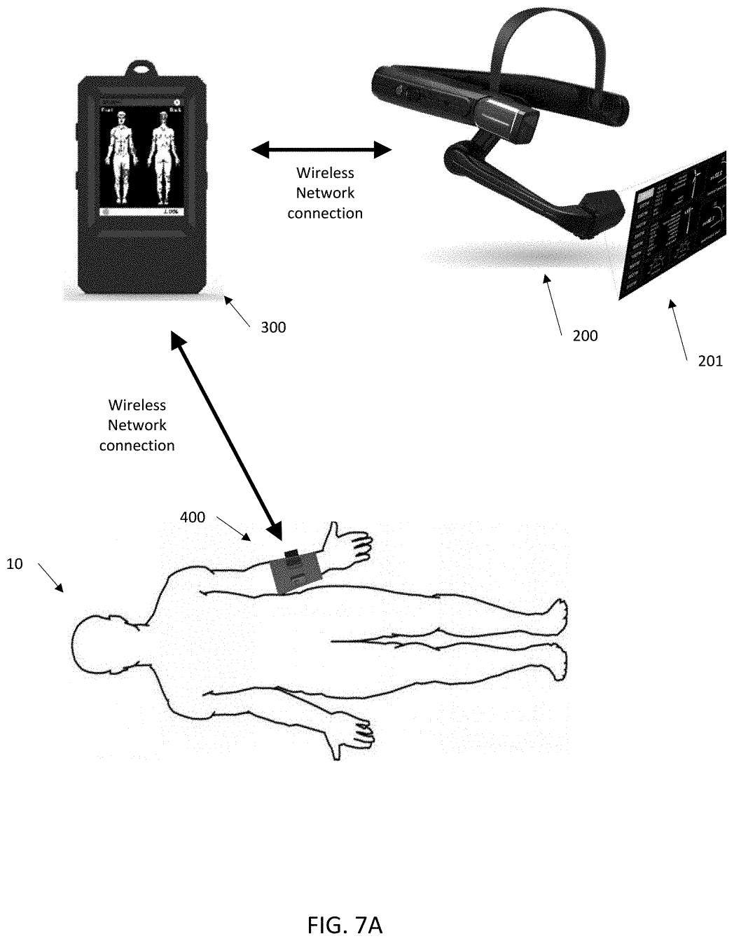

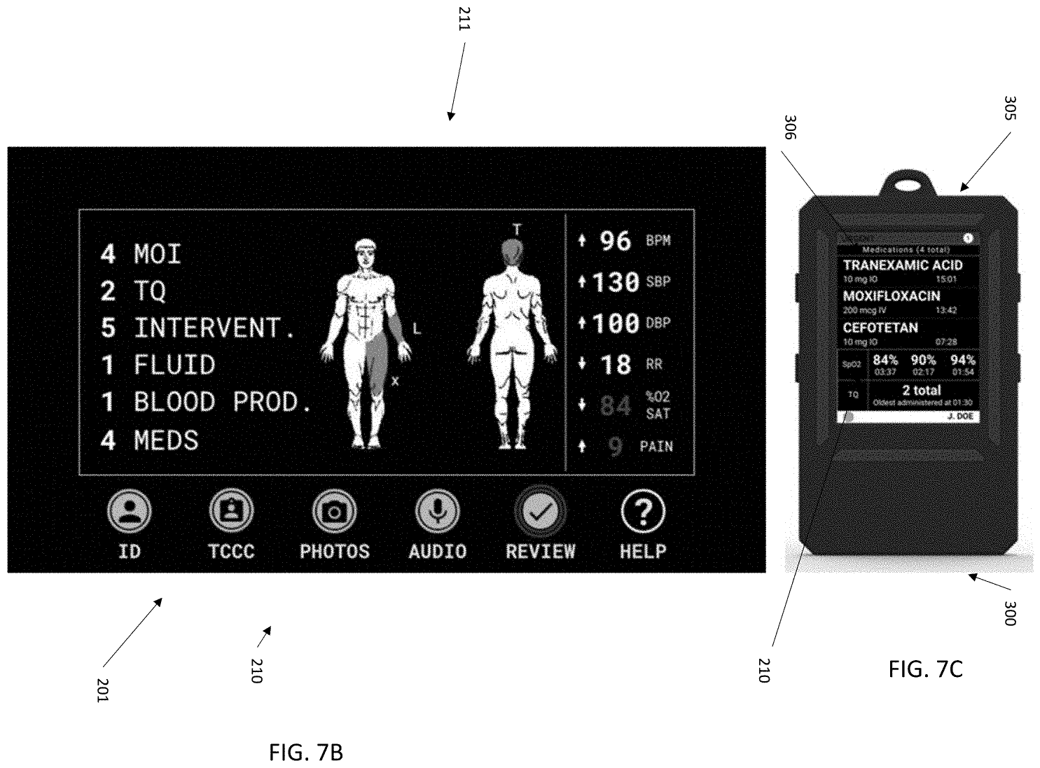

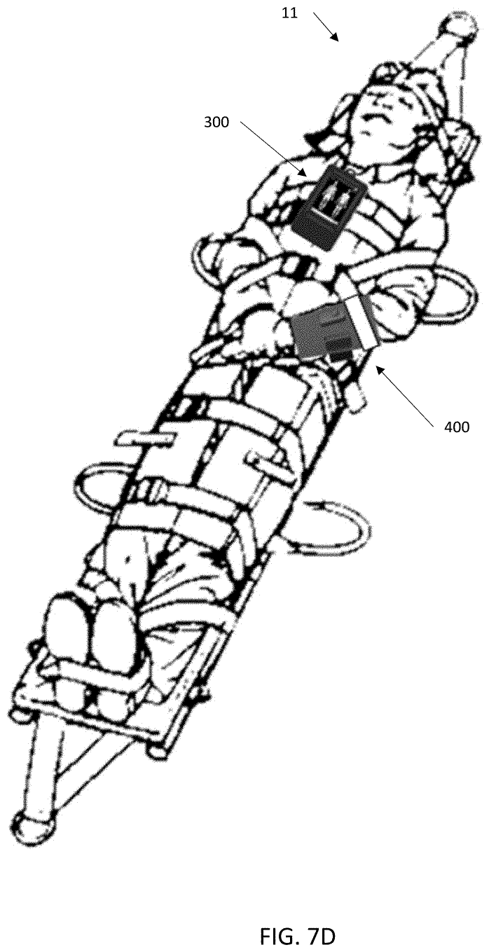

The system shown in FIG. 7A illustrates the network connections for the tag, PMSU, and HMD described above. Patient vital signs are sensed and recorded by the PMSU. These data are then communicated to the data tag for storage and display through a wireless network connection. The HMD can communicate with the data tag for review, retrieval, and control of the PMSU. Alternatively, the HMD could communicate directly with the PMSU. The data tag and the PMSU could be connected by a wired data connection such as universal serial bus USB or other connection, or alternatively by a wireless connection such as Bluetooth or wife.

The system shown in FIGS. 7B and 7C depicts the caregivers view of the patient status on the data tag 305 and an HMD patient status page 201 showing a patient avatar 211 illustrating patient information 210 such as injury locations, treatments such as tourniquets that have been applied, chest tubes applied, dressings applied, or other treatments, and vital signs transmitted from the PMSU. The data tag 300 is shown displaying medicine administered 306 to the patient which has been transcribed by the caregiver though the HMD and recorded on the data tag. Also displayed on the data tag are patient vital signs 307 and triage status through the patient status lights.

The systems shown in FIG. 7D shows a patient being evacuated wearing the PMSU and the data tag. The data tag is shown affixed to the outside of the patient's wrapping, to be visible to downstream clinical staff for evaluation of the patient's status and history without dislodging the wrapping. The PMSU is shown on the patient's forearm but may be placed in other locations where direct skin contact is possible.

Alternative embodiments of the PMSU may also be affixed to the skin with an adhesive patch.

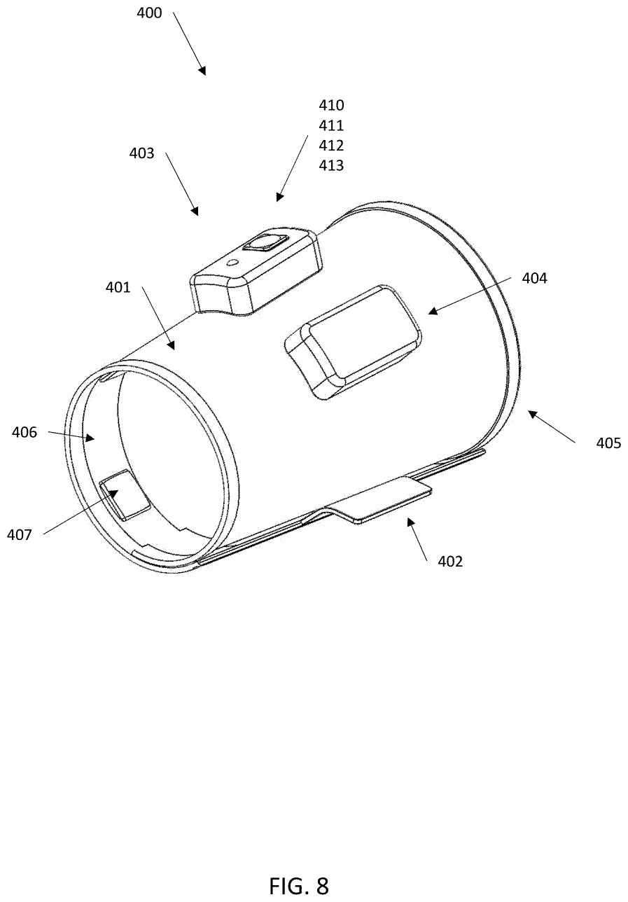

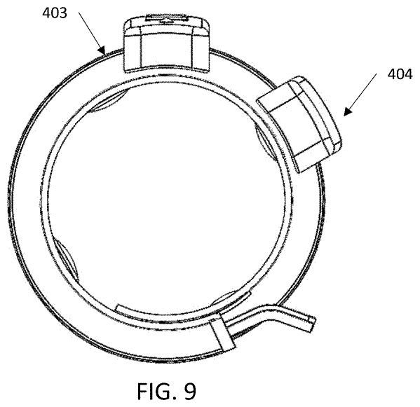

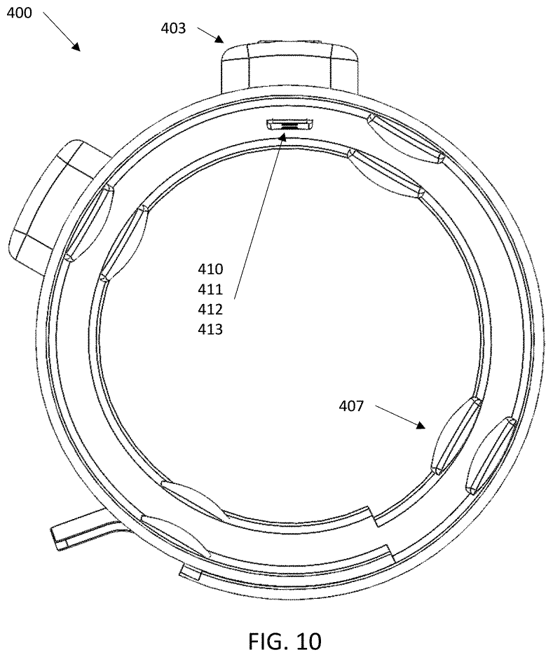

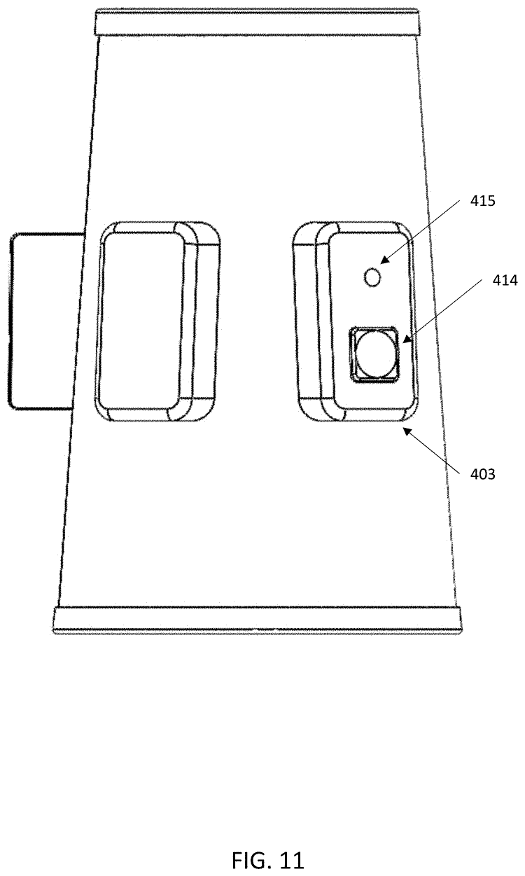

The PMSU system shown in FIGS. 8-11 is a flexible fabric sleeve 401 which is adjustable in size through a hook and loop adjustment strap 402 for adjusting the circumference of the sleeve to ensure patient contact with the physiological sensors 407. The PMSU includes one or more electronics housings 403 activation switches 414, status lights 415 and battery packs 404 which are connected to one or more sensors 407. The sensors are connected via flexible circuitry which is incorporated into or on the fabric sleeve. for enclosing the electronic components required to read the sensors. The PMSU may further contain one or more regions of higher or lower elasticity 406 for ensuring skin contact between the sensors and the patient. The electronics housing 403 includes physiological sensors which do not require significant spacing to achieve high quality signals. Examples of such sensors are PPG 410, HR 411, Temperature 412, GSR 413, or other sensors. The sleeve further incorporates an array of sensors (407) which are distributed throughout the interior surface are of the sleeve. The distributed sensors physiological monitoring which require the sensors to be spaced apart to acquire a high-quality signal. Examples of such sensors are ECG or blood pressure when measured by pulse transit time measurement techniques. ECG sensors which are also called leads or electrodes are in contact with the patient's skin for measuring voltage associated with heartbeats. These sensors may be dry or may be covered in an coating such as conductive gel. Electrodes are typically made from a conductive material such as silver chloride AgCl or other conductive material. ECG signals are particularly sensitive to having the leads placed far apart, to generate a large enough signal to noise ratio to produce a high-quality signal. In this example a single lead ECG is shown with one electrode array near the patient's wrist and second electrode placed further up the arm. Another sensor placement would be for the first sensor to be placed on the wrist/shoulder or wrist/torso combination. An example of such lead placement is shown in FIG. 11 ECG leads (450) are connected vial wires 451 to the PMSU. ECG lead scenarios compatible with this system may be a single lead, or multiple lead ECG such as a 3 lead or a 12 lead ECG electrode placement scenario. Also shown in FIG. 11 is a finger base pulse oximeter 460 which clips onto the patient's finger.

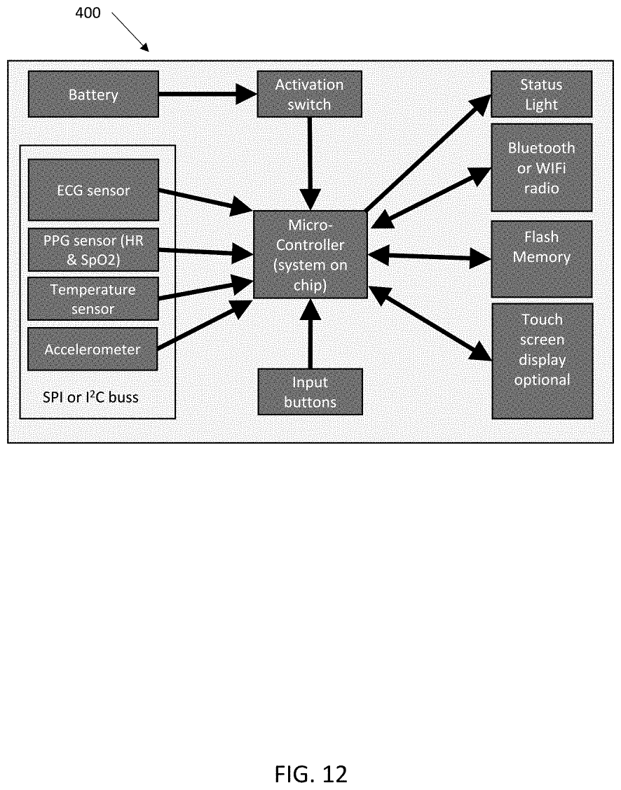

FIG. 12 is a schematic of the components incorporated into some embodiments the PMSU. The PMSU can be configured to include a microprocessor to manage data flows. A Bluetooth radio communicates with the tag or HMD to send and receive data to and from the tag and the HMD. Due to the generally close proximity between the tag and the PMSU, the battery power required to maintain the wireless connection may be reduced. The data tag may be configured to have a larger battery for data storage and for data transmission from the patient centric network to other stakeholders or to cloud computing systems. The PMSU wireless radio may also send and/or receive data from the hospital's EHR once the patient and the tag are transported to the hospital. The PMSU is configured to include non-volatile storage for recording the patient's health records. The PMSU also incorporates an A/D converter and/or amplifier for recording patient ECG voltage measurements. Additionally, the PMSU may incorporate one or more of the following: PPG sensor, temperature sensor, GSR sensor, accelerometer, blood glucose sensor, blood pressure cuff, blood pressure sensor via pulse transit time, or other physiological sensors for collecting physiological data as defined above.

The PMSU may also be configured to include a low-cost display for communicating health data to caregivers without data tag or HMD hardware. An internal battery powers the PMSU and a status light may be configured to indicate the device is on or provide patient status similar to the data tag described herein. The HMD of the caregiver may also be replaced with a smartphone, smart watch, or other personal computer with the ability to receive patient information from the caregiver, communicate with the data tag, and/or display information to the caregiver.

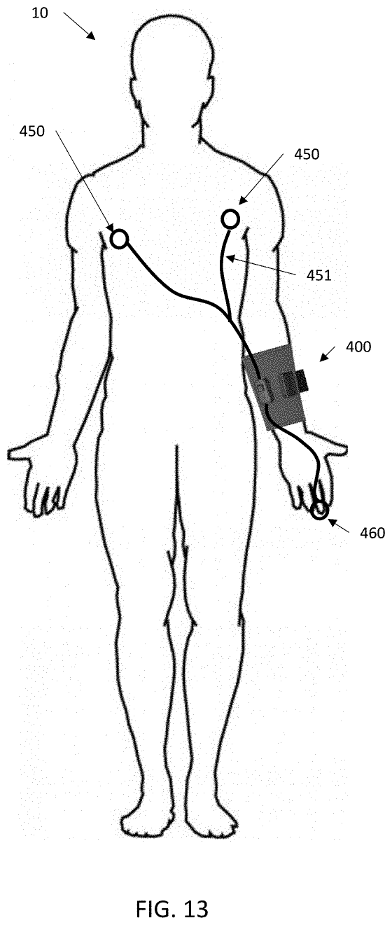

The PMSU system of FIG. 13. shows the addition of additional sensors to the wristband monitor. ECG electrodes 450 connected by wired connection 451 may be attached to the PMSU for providing increased sensitivity and/or accuracy of the ECG. Wrist based ECG measurements are enhanced with additional electrodes for generating a higher fidelity electrical waveform of the electrical activity of the heart. Two additional electrodes are shown here, but the system may be capable of using up to 12 electrodes. Also, an external SPO2 sensor may be attached 460, for providing additional pulse oximetry data to the PMSU.

Referring back to FIG. 7A, the PCN system includes the HMD 200 on the caregiver providing treatment. The display of the HMD provides a view 201 similar to looking at a virtual 7 inch tablet at arm's reach. The data tag 300 communicates wirelessly with the HMD and the PMSU 400 to collect and integrate patient data. The tag transports with the patient throughout the continuum of care, recording treatments and vital signs for review by caregivers. Once the patient is stable the caregiver may disconnect the HMD from the tag to care for other patients. The tag will continue to monitor the patient and will alert the caregiver to patient status changes. The PCN system tag combines the available physiological monitor and/or treatment data provided by the HMD and the PMSU to create provide a local clinical decision-making support (CDMS) score which will provide a unified view of the patient's status. (CDMS) tool algorithms indicates if a patient is experiencing cardiopulmonary arrest, shock, and/or hypothermia. The CDMS may provide recommended treatment methods or recommend a patient status change to provide more urgent care to the patient. The data tag will provide a visual indicator (on the screen display) or on the HMD, of when a patient is exhibiting signs of hemodynamic instability from cardiopulmonary distress, circulatory shock, or hypothermia.

An alternative embodiment of the PMSU may integrate the functionality of the medical records data tag and the PMSU into one unit. The display, battery, patient status lights, microphone, input devices, memory, processor, and wireless communications radio may be integrated with the medical sensors of the PMSU into a single arm band unit.

CDSS and CMDS Tool Flow Charts

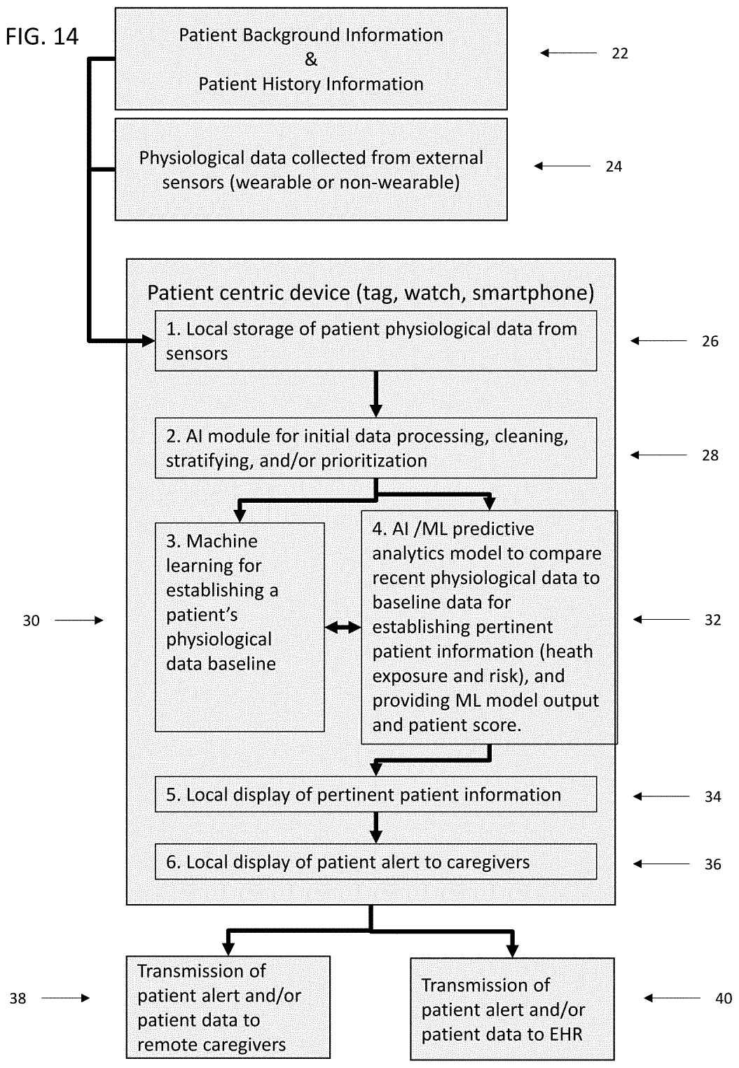

The CMDS tool shown in FIG. 14 depicts a flowchart which provides the ability of the system to calculate a patient CMDS score while processing the data on-device. As described above, the system can include a and a PMSU 400 equipped with physiological and/or environmental sensors, an HMD 200, an electronic patient record tag 300 or smartphone. At step 22, patient background information and patient history information can be taken by a provider or accessed from an electronic medical history for the patient. In some embodiments, an assessment of the patient can be taken in real-time (such as by a provider with a HMD 200). At step 24, physiological data can be collected with external sensors that can be wearable or non-wearable, such as with the PMSU 400 described herein. Next, at step 26, the patient physiological data can be stored locally, such as on an electronic patient record tag 300. At step 28, the tag, or any other local/patient centric device, can include an artificial intelligence (AI) module configured for initial data processing, cleaning, stratifying, and/or prioritization. Next, at step 30, the patient centric device can execute machine learning software and/or algorithms to establish the patient's physiological data baseline. At step 32, an AI model can be used for comparison of recent physiological data to baseline for establishing pertinent patient information and a CMDS score. At step 34, the CMDS score is then processed for local display of patient condition to the patient and/or local caregivers, such as with onboard patient status lights or a display, or on local caregivers devices (such as the HMD). Additionally, at steps 38 and 40, the patient status score can be transmitted to remote caregivers and to an EHR for review and patient intervention/treatment if needed.

For ML model development, datasets pre-processing is conducted for static and dynamic analysis, using cross-sectional and vital sign data, respectively.

Cross-Sectional Data Pre-Processing for Mortality Risk Prediction:

For mortality risk prediction, cross-sectional data can be pre-processed to classify patients as belonging to either “discharged” or “deceased” outcome groups. Only patients from these outcome groups with a confirmed infection, for a virus such as COVID-19, can be included in the dataset. Features can then be selected using a Student T-test with 99% confidence. Patients missing all vital sign data can be removed, and remaining missing values can be input using the average of all patients in the data set.

PostgresQL can be used to extract features from the dataset for each patient and to calculate values used to fill in missing data. The final PostgresQL table can then be exported as a .csv file. Python with the Pandas library used to import and clean the data, using Numpy for the latter. All other data preparation (shuffling, training/validation split, etc.) and model training can be done using the Scikit-learn library.

Cross-sectional data Features: For mortality risk prediction, model features can include items such as age, gender, and vital sign measurements in which there was a statistical significance between cohorts (discharged vs. deceased). Each patient can be represented by a feature vector composed of 21 different features: age; gender; first and last measurements taken per patient during a treatment period for SpO2 and diastolic blood pressure; average, minimum and maximum values for systolic blood pressure, diastolic blood pressure, temperature, heart rate, and SpO2.

A second variation of the dataset can be created by augmenting the above feature vector with a binary feature, in which 1 or 0 was assigned depending whether or not the patient needed mechanical ventilation.

The cross-sectional data split can include, for example, 70% training data and 30% validation.

Time-Series Data Pre-Processing for Mortality Risk Prediction

Included/Excluded patients: Patients included in the pre-processed dataset for mortality risk prediction can be the intersection of patients included in the pre-processed cross-sectional data set, and those that contain vital sign measurements in the raw dataset. Excluded patients can be those in a sub-population for a specific disease state. For COVID-19 for instance, patients can be excluded who are 20-years-old and younger because measurements may be missing for the entire sub-population. Furthermore, patients with no vital sign measurements may not included in the pre-processed dataset.

Features: Vital sign data for the following features can be included as time-series data for mortality risk prediction: maximum/minimum blood pressure values, heart rate, O₂ saturation, and temperature. The blood glucose feature may be removed as it can contain all zero measurements, and the observed O₂ saturation may be removed because this feature is categorical and sparse.

Data Imputation: Missing patient measurements can be input with previous day measurement for the same patient if available. Otherwise, the median measurement for that patient across all available measurement dates can be used. Finally, if no measurement was taken for a patient for a specific feature, the value may be input using the median of the sub-population feature values, where the age-defined sub-populations were consistent with those generated for cross-sectional data imputation.

Missing measurements may be input to maintain a constant sampling interval to compensate for irregular sampling. These measurements can be input up until the discharge date from inpatient care. Mortality risk can be predicted at each time step with target replication for all time steps, where 1 indicates death and 0 indicates discharge.

Individual .csv files can be generated such that patients were listed in increasing ID order and the time-series data were sorted by date and time. One of each of the following may be generated: static (cross-sectional), and dynamic (vital signs), and labels. If multiple vital sign measurements are present for the same day, the most complete measurement (time step with the most non-zero measurements) can be used.

PyTorch Dataset: PyTorch provides two data primitives that allows pre-loaded datasets to be used with new data. Specifically, the PyTorch dataset prepares static and dynamic data for input into the sequential model; groups patient ID with features and the assigned label; extends capability to both static and dynamic data, to be used with ensemble model; and stratifies the training/validation/testing split by class label to account for class imbalance. Stratifying the dataset ensures that (i) each subset has the same distribution of class labels for consistent training and evaluation data, (ii) a 10% held-out test set to evaluate final model, and (iii) 80/10% training/validation K-fold cross-validation to assess the robustness of the model to selected training data.

Data Pre-Processing for CB Detection

In the example provided (below), SARs-CoV-2 is described as the biologic agent. A similar methodology could be applied to other infectious diseases and CB agents.

Data Interpolation. Patients can be included in the pre-processed data set (COVID-19 positive, COVID-19 negative). For each patient, two intervals can be defined: an illness period during which the patient had COVID-19 and a windowed interval containing data input into the sequential model. For COVID-19 positive patients, the illness period can be defined as two weeks prior to the symptom onset date until the recovery date and is used to label these included time steps as COVID-19 positive. For COVID-19 negative patients, the illness period may be defined as the time between symptom onset and recovery. The windowed interval may include patient data from one week before the illness period to one week after the end of the illness period.

Patients who may be missing symptom onset/diagnosis/recovery dates can be assigned dates using a standard interpolation method. For COVID-19 positive patients missing symptom onset dates, the illness period may be designated to start two weeks prior to the diagnosis date. For COVID-19 positive patients missing recovery dates, the illness period can be designated to end one week after the diagnosis date or 1 week after the symptom onset date, depending on which date is available. For COVID-19 negative patients missing recovery dates, the illness period may be designated to end two weeks after symptom onset. Finally, for the single COVID-19 negative patient missing all dates, a random sample of 4 weeks can be used to window the patient's data.

Data Pre-Processing. Heart rate outlier measurements can be removed from the data set. These may include data points that have a heart rate above 200 bpm or below 30 bpm. That dataset indicates that heart rate measurements were captured at irregularly-spaced intervals (but typically once every 15 seconds). To reduce the number of data points into the model, the data points can be down-sampled within the windowed interval by taking the median measurement for each day in the sequence. This may be necessary because sequential models have a tendency to “forget” the information learned at the start of the sequence if the input sequence is too long. By restricting the input sequence to be <100 days long, the important daily information can be preserved.

Since labels are assigned by day, for COVID-19 positive patients, the data points within the illness period can be labeled as positive and all other data points can be labeled as negative.

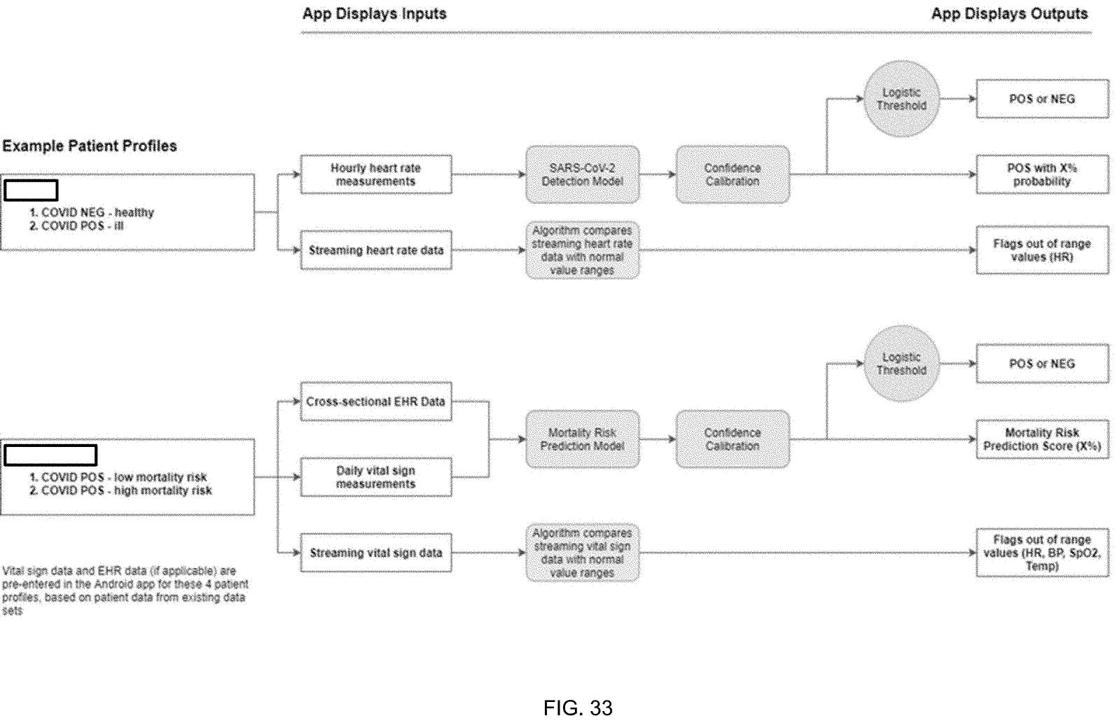

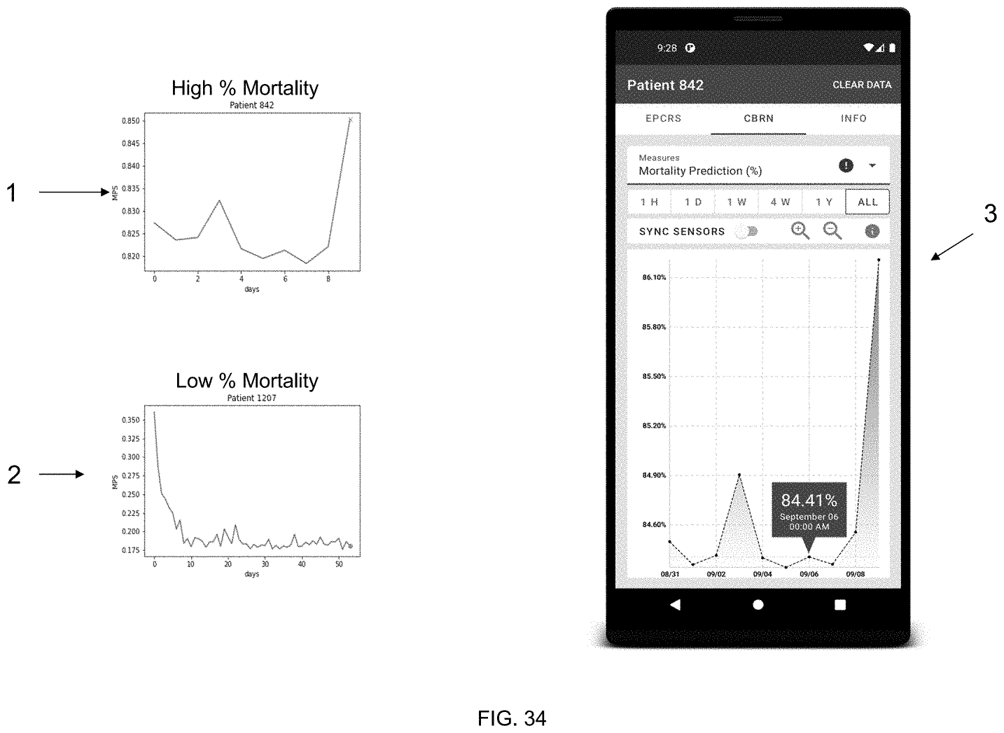

In reference to FIG. 33-34, to apply the machine learning models in clinical use, machine learning model output data may to converted for 2D or 3D visualization displays. For instance, for CB detection (for COVID-19), the smartphone application provides an hourly binary result of COVID positive or negative, depending on whether a patient's output probability is greater or less than the logistic threshold, respectively. For mortality risk, the smartphone application provides a mortality risk prediction score as a function of time (in days). The application also compares streaming vital sign data with normal value ranges, and flags vital signs that are out of normal clinical range.

In reference to FIG. 34, a patient's mortality prediction score from the machine learning model output data 01 corresponds to the screen shown on the smartphone display 03 for a patient with a high risk of mortality.

The aim of the mortality prediction ML model, shown as an example in FIG. 34, is to generate daily mortality risk predictions for COVID-19 positive patients based on basic vital sign measurements over time. EHR cross-sectional and time-series data was used to train the model, consisting of COVID-19 positive patients, with their vital sign data from admission to discharge dates. The machine learning model is a recurrent neural network (RNN) voting ensemble model that takes a combination of static and dynamic features as input, with daily time steps for the vital sign sequences, and outputs a daily prediction score for mortality risk where a high score indicates high likelihood of mortality. The model's output data provides a mean and standard deviation of the evaluated metrics across 10-folds of cross-validation and is an evaluation of the model's prediction for each patient's final time step compared to ground truth label of discharge or mortality. A validation sensitivity and specificity above a designated threshold (i.e. in this case 80%), showcases the model's ability to correctly identify patients who have a high mortality risk as well as those with a low mortality risk.

In reference to FIG. 31, the aim of the chemical-biological agent detection ML model (for COVID-19 as a specific use case) is to generate hourly predictions for COVID-19 exposure based on a singular time-series input (in this case heart rate measurements). Heart rate data from a commercial smartwatch was used to train the model, sampled at a frequency of approximately one measurement per 15 seconds. These data were collected from COVID-19 negative and 32 COVID-19 positive patients and the heart rate sequences span both “healthy” (unexposed) and “μl” (exposed to COVID-19 or another illness) periods for each patient. The machine learning models investigated include an LSTM recurrent neural network (RNN) and an RNN voting ensemble, both of which take a single dynamic feature, averaged hourly, as input and outputs an hourly prediction of COVID-19 exposure

The ML model output displays or CDMS score may be calibrated to provide patient-specific performance evaluations (i.e. evaluation metrics computed based on each patient's prediction), or global performance evaluations (i.e. average metric over all time steps in a patient's sequence for all patients).

At step 34, the CMDS score is then processed for local display of patient condition to the patient and/or local caregivers, such as with onboard patient status lights or a display, or on local caregivers' devices (such as the HMD). Additionally, at steps 38 and 40, the patient status score can be transmitted to remote caregivers and to an EHR for review and patient intervention/treatment if needed.

A CMDS score according to some embodiments can be an assessment of the patient's health. In some embodiments, the CMDS score can be used to indicate if a patient is exposed to or infected with a communicable or infectious disease or virus (such as the flu, COVID-19, etc.). The CMDS score can be used to indicate if a patient has been exposed to a chemical or biological agent, and the health risk associated with the exposure. The CMDS score can simply be an output that indicates a positive infection or no infection; positive exposure or no exposure. In other embodiments, the CMDS score can be an assessment of the patient's mortality risk, a prediction of a patient's severity of injury or the probability of developing a secondary injury from an initial injury or exposure. The assessment of patient risk can be tied to a positive or negative infection/exposure determination, or can be unrelated to a prior diagnosis. In some embodiments, the CMDS score can be displayed as a percentage, with 0% being a very low risk of a specific outcome (such as mortality) and 100% being a very high risk. It should be understood that other output systems can be used, such as population-level and patient-specific statistics, as long as they convey the health risk associated with a particular clinical condition.

In some embodiments, the CMDS score can provide a triage or care recommendation for a medical provider or caregiver. For example, if the CMDS score indicates a high mortality risk or determines a diagnosis of a serious infectious disease or chemical and biological (CB) exposure, the CMDS score may additionally recommend that the patient receive immediate care. This can be provided in the form of recommending treatment according to one of the known triage systems, such as START Triage, SALT Triage, or JumpSTART. For example, a patient with a high mortality score but one that would respond well to treatment may be given a treat immediately score or indicator.

In military and civilian applications, the CDMS score can be used to assist combat medics, physicians, and first responders in rapidly identifying casualties with CBRN agent exposure, to reduce errors in diagnosing a casualty with CBRN exposure, and to reduce the amount of training required by care givers to identify casualties with CBRN agent exposure. For emergency medical services (EMS) and in-hospital operations, the CDMS score could integrate with existing medical platforms to support in-hospital clinical documentation and decision support.

In some embodiments, the CMDS score can provide predictive analytics for clinical areas, including but not limited to the following areas: to predict the onset of sepsis for the management of CBRN injury, to predict acute respiratory failure (ARF) and acute respiratory distress syndrome (ARDS) from infectious disease or exposure to CB agents (such as COVID-19); to predict the presence/absence of head injury, injury severity and mortality due to a traumatic brain injury (TBI), blast pressure, or head impact; to detect neurological dysfunction due to COVID-19 and to other CB exposures and/or neurological conditions; to detect the presence or absence of a concussion and associated risks; to detect potential heat exposure and heat stroke; to predict ocular and musculoskeletal injury; and to predict chemical burns from CBRN exposure.

Machine learning model outputs from the disclosed system can provide contextual information through developing a predictive analytics system that “learns its patient.” This uses mathematical models to provide useful readiness information from real-time assessments combined with personal contextual/temporal data (i.e., time-series and cross-sectional data). These models can provide automatic decision support for an individual patient in the context of his or her real-time physiological status and CBRN exposures.

The ability to predict CBRN exposures previously relied on visual inspection and on generalized models based on estimated inputs about individuals and ambient conditions. Wearable physiological monitoring can now provide predictions about an individual's health and performance from a patient's real-time physiological state. An extension of this is a provision for “shared sensing.” For the military, this includes enhanced environmental awareness to provide the physiological status of all members of a small group or unit, thus allowing the unit to perform as a single entity.

The system disclosed herein can impact the development of novel visual interfaces, for 2D (heads-down) and 3D (heads-up) displays to visualize the results of CBRN exposure and detection ML algorithms and health risk/injury prediction models.

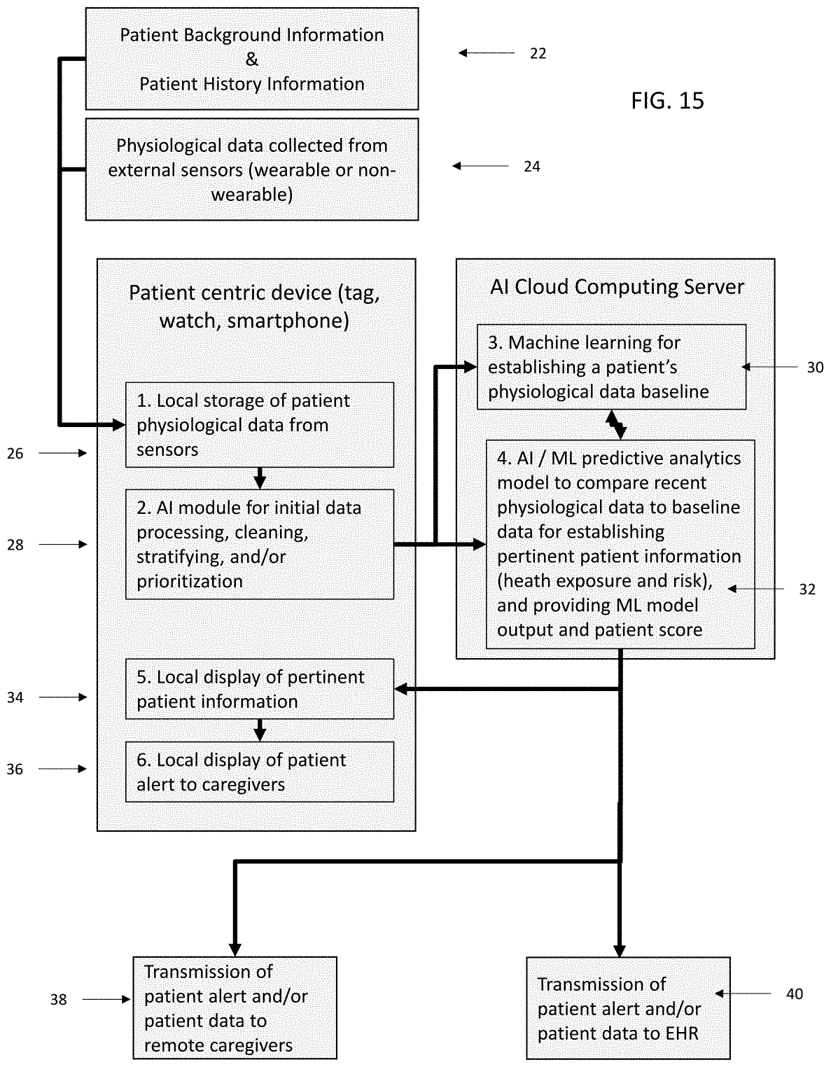

The CMDS tool shown in FIG. 15 depicts a flowchart which provide the ability of the system to calculate a patient CMDS score while processing the data on a remote cloud server, transmitting the CMDS score back to the data tag for display. The flowchart in FIG. 15 is similar to the flowchart of FIG. 14, except in this embodiment, steps 30 and 32 are processed on a remote server, instead of being processed locally as in the embodiment of FIG. 14.

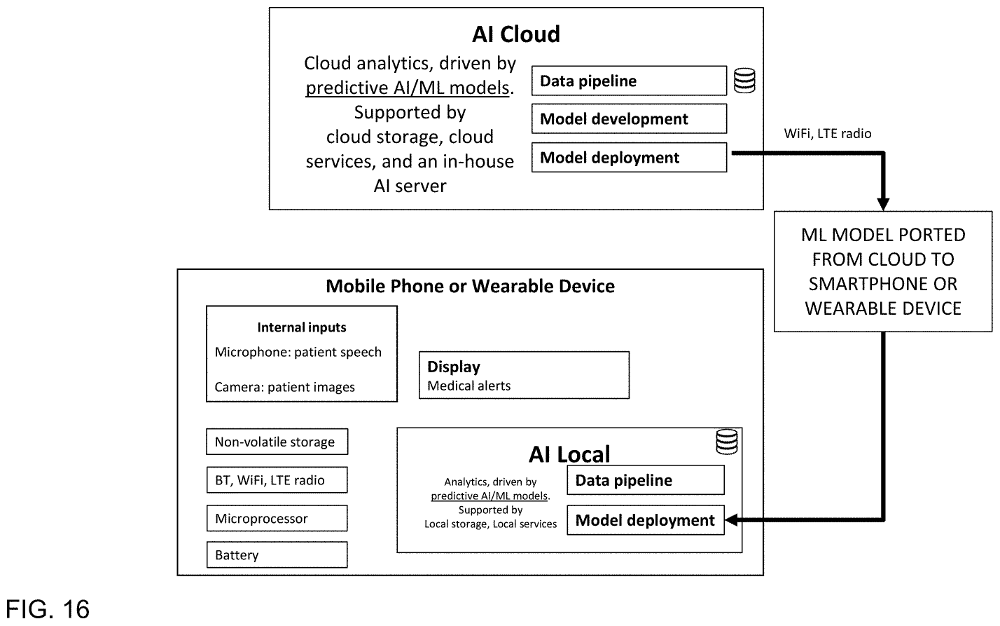

Referring to FIG. 16, Clinical decision support systems (CDSS) applications can be deployed to mobile devices with pre-trained AI models for offline use. Referring to FIG. 16, AI model development can be done on the cloud, with cloud analytics driven by predictive AI/ML models. This process can include a data pipeline, model development, and the model deployment to other devices. In this embodiment, the machine learning model can be ported from the cloud into the device, and input data can then be processed on the local device using the AI model locally. The output of the AI model may be a CMDS score for use in the CDSS application. A CMDS score may be compared to a baseline CMDS score for each patient, and alerts provided to clinicians once a threshold or data window has been exceeded.

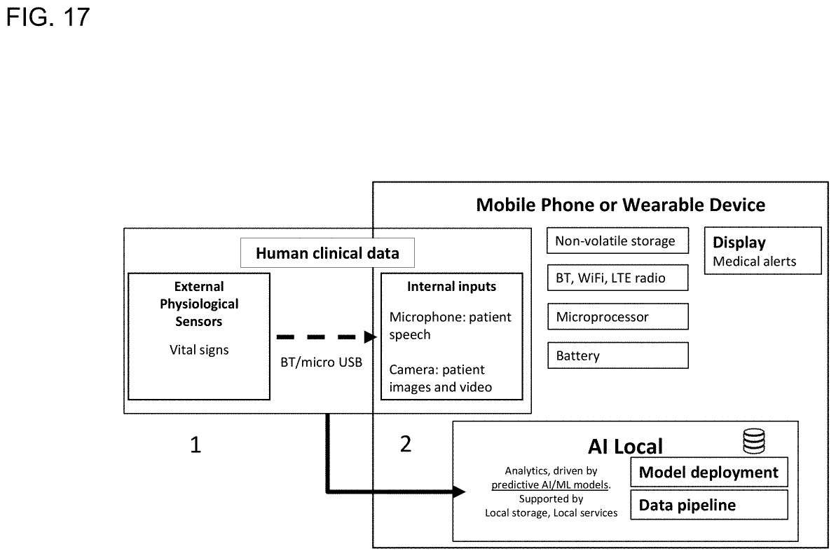

Referring to FIG. 17, Data Inputs include data from external physiological sensors (such as PMSU 400) sent to the local AI model, running on a mobile phone or other wearable device (such as electronic patient record tag 300). Data can be sent from a local device or wearable (speech, images) to the AI Local model running on the mobile phone or wearable device. In some embodiments, data is captured by the different inputs to the local device. Internal inputs can include the local device's microphone and/or camera, which can be used to capture user speech and images.

Additionally, the local device can be connected to external physiological sensors for capturing patient vital signs. This information can be used by CDSS apps for offline prediction/inference by any attached AI models. In offline use, this is how the local device would be used for AOD running offline. Processing in this embodiment can be performed locally, with no cloud computing needed.

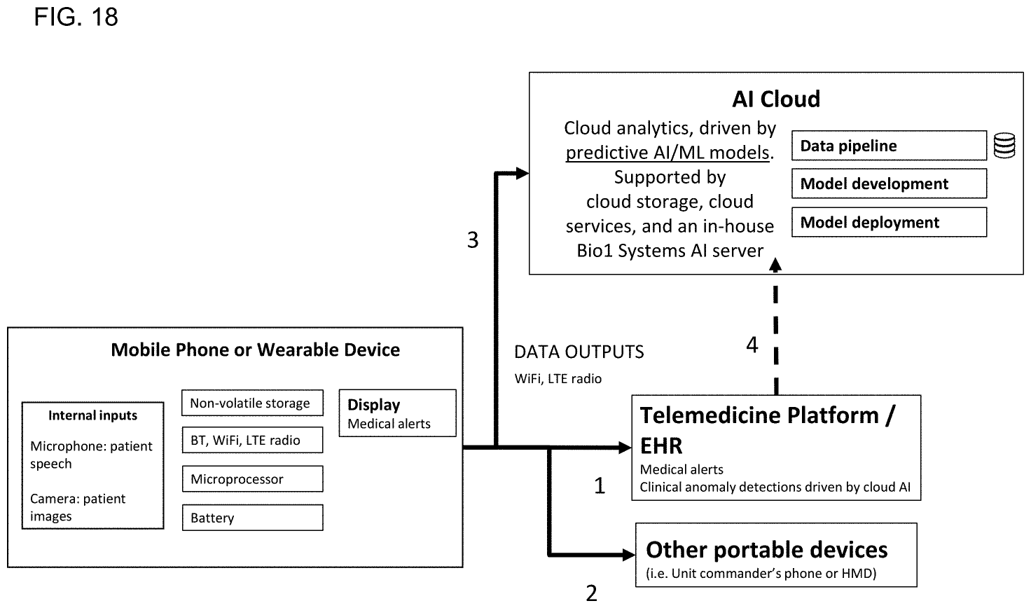

Referring to FIG. 18, after clinical data capture has occurred and/or when internet connectivity is regained, all captured data (raw and/or processed) can be uploaded from the mobile phone or wearable device directly to an AI cloud or indirectly via the patient's EHR platform. This is how the data flows from the offline environment into the online EHR. The local device such as a mobile phone or other wearable device can be configured to output the ML model results/medical alert from the AI cloud to a number of other devices, including to 1) other portable devices, 2) to a telemedicine platform or a patient's EHR, and/or 3) to an AI cloud server. On the AI Cloud, analytics are driven by predictive AI/ML models, cloud storage and services, and include a data pipeline, model development and model development.

Model outputs from the AI Cloud to other portable devices, a telemedicine platform or a patient's EHR provide a continuous machine learning loop that iteratively improves as it learns a user's or patient's performance characteristics.

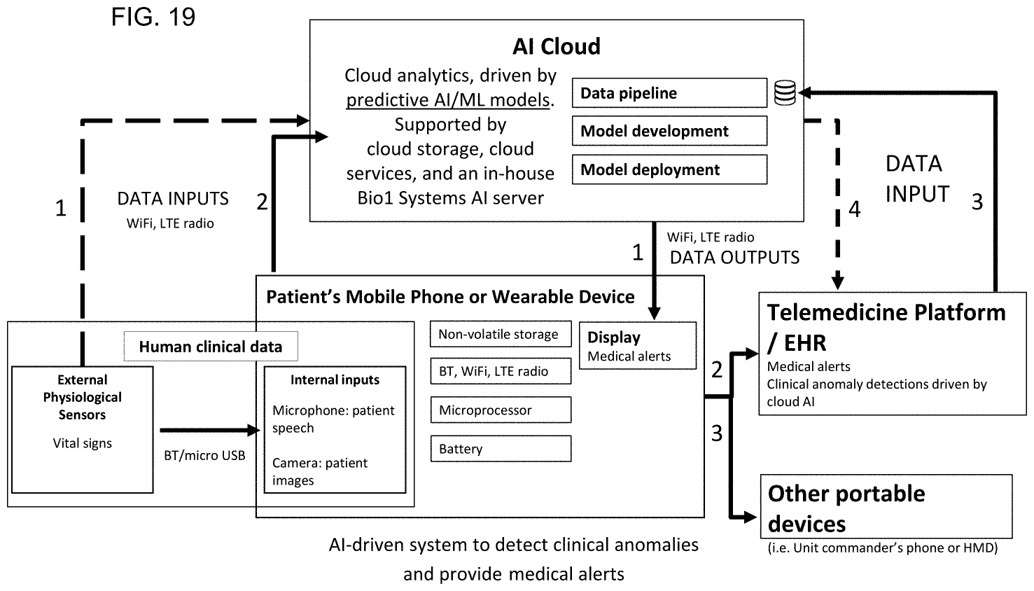

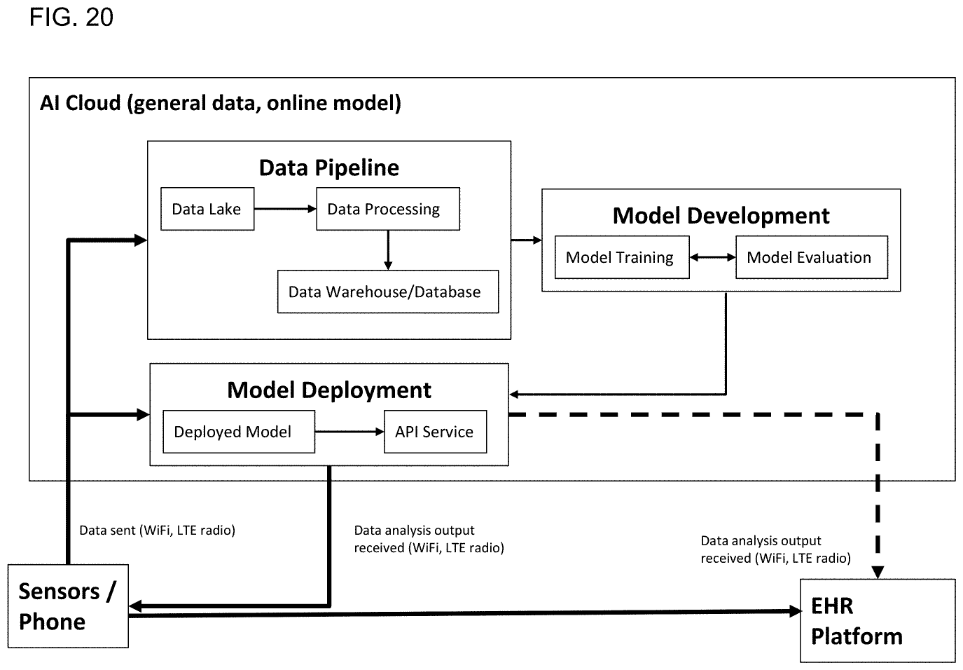

Referring to FIG. 19, an AI driven system is shown which describes the transfer of data from various sources into the AI cloud for model development and deployment. Inputs into the AI cloud can include 1) human clinical data such as physiological monitoring sensor data or environmental data from worn devices, 2) sensor data and internal inputs (speech, images) from a smartphone or other wearable devices, and 3) clinical data from a patient's EHR. Referring to the embodiment of FIG. 20, however, it can be seen that in some embodiments there is no input into the AI cloud from the EHR. Data outputs from the AI cloud can include: ML model results/medical alerts sent to the smartphone or mobile device. Additionally, as described above, ML model results/medical alerts can be sent directly from the smartphone or mobile device sent to a patient's EHR, or to other portable devices. When the patient's mobile device has internet connectivity, automatic, manual, asynchronous, and real-time data exchanges are all supported to the AI cloud and EHR platforms. The AI model can be static or can be evolving as additional data flows through the model. In this case the model can undergo continual training and will improve the more the system is used.

In some embodiments, referring to FIG. 19, data capture can occur simultaneously with data analysis. In other embodiments, the data capture can be asynchronous. The main difference is that asynchronous data capture occurs at a time prior to data analysis. Asynchronous data capture use could also apply to offline use, such that data is collected and stored on a smartphone or mobile device, and then processed in the cloud at a later time.

Use case example: Vital signs are captured for the patient automatically by external physiological sensors connected to the phone over a Bluetooth/wifi/or wired USB connection. The patient or caregiver also manually records their speech and takes images relevant to their medical state. The phone sends all captured patient data to the cloud. The instant response by the cloud to the data received is to use an AI model to generate and send analytics back to the client device. The analytics may include a CMDS score for comparison to a patient's baseline. The cloud can also send this information directly to the EHR. The client device then reads the analytics and displays any important medical alerts or information as part of the CDSS. However, the cloud also stores the data for future processing and further refinement to existing AI models. The EHR receives any processed data from the patient's mobile device directly or indirectly through the cloud. Clinicians with access to the EHR may also push data to the platform that the cloud can subscribe to for updates.

In the embodiments above, clinical data is typically automatically entered into the patient's mobile phone or wearable device, to run ML models either locally on-device or to run models from the AI cloud. In other embodiments, clinical data is manually entered into a patient's mobile phone or wearable device and transferred to the AI Cloud, to run models on device or via the cloud. Clinical data may also be transferred from a patient's EHR to the Biol Systems AI Cloud. No direct link from physiological sensors to cloud. Data Outputs Include: ML model results/medical alerts from Biol Systems AI Cloud to the smartphone or mobile device. ML model results/medical alerts from the smartphone or mobile device to a patient's EHR. ML model results/medical alerts transferred from the smartphone or mobile device to Other portable devices. ML model results/medical alerts sent directly to a patient's Telemedicine platform/EHR (less common data transfer route). Manual data entry involves data that has been manually entered into a smartphone or manually entered into a patient's EHR by a clinician and sent to the Biol cloud. It would not be used as input for real-time data analytics.

Referring to FIG. 20, this diagram outlines the general process for training AI models from data captured on mobile devices for providing the patient's EHR platform with insights into their health data. Data captured by the mobile device can be fed into the AI cloud, which can include a data pipeline (with a data lake, data processing, and a data warehouse/database), an AI model deployed for instant inference/prediction over the observations. The data pipeline (a pre-processor for organizing the data and cleaning the data for later flow into the model development) stores all uploaded data in a data lake (some space on the cloud for unstructured data), which is a collection of the raw unstructured data for later processing and provides raw data to a processing service which prepares the data for model development, and stores processed data in a warehouse/database. Model development uses processed data to train AI models, and evaluates trained AI models and uses evaluation results to refine the model in an iterative process. Once a model has been developed the model can be deployed on a cloud server and the model can be exposed to client devices through an API to provide detection/inference. Output from deployed models is then sent to the phone and transferred to a patient's EHR. Alternately (but less common), data is sent directly from the AI Cloud to a patient's EHR. Data types that are used in this process are: Time series data: blood pressure, heart rate, temperature, SP02, Spirometry data, nerve agent ecg measures. This time series data can be all over the time domain. This process can also use other cross sectional series data that could be used in this processing, including wound assessment/healing, TBI classification, chem/dx, cholinesterase (not time dependent data sets).

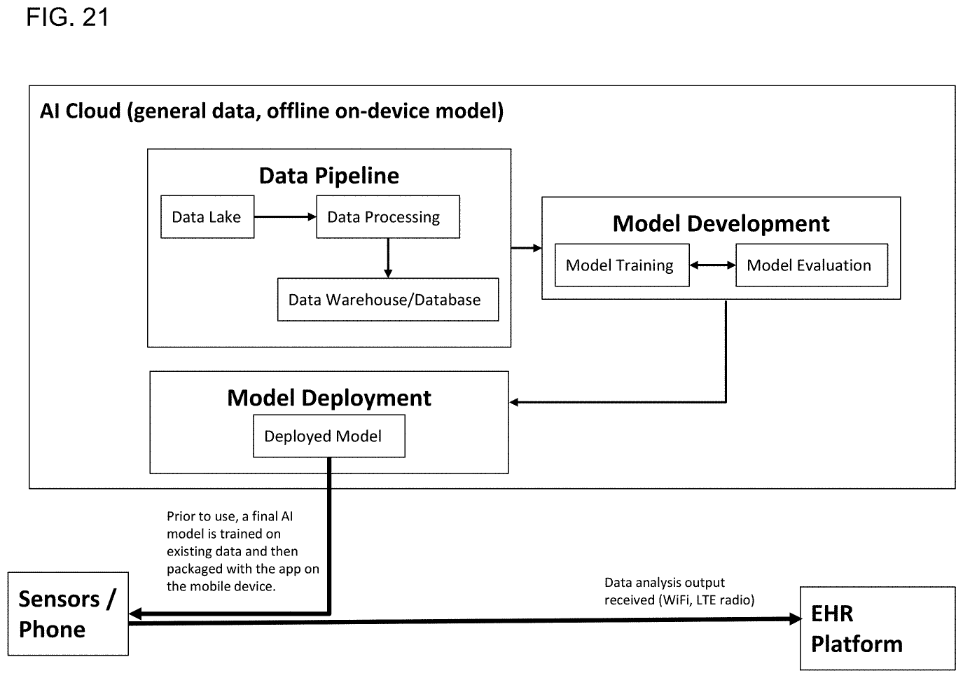

Referring to FIG. 21, this diagram outlines the general process for training AI models from data captured on mobile devices for providing the patient's EHR platform with insights into their health data. Data captured by the mobile device can be fed into a data pipeline and a model deployed on the cloud for instant inference/prediction over the observations. As described above, the data pipeline (a pre-processor for organizing the data and cleaning the data for later flow into the model development) stores all uploaded data in a data lake (some space on the cloud for unstructured data), which is a collection of the raw unstructured data for later processing and provides raw datato a processing service which prepares the data for model development, and stores processed datain a warehouse/database. Model development can use processed data to train AI models, and evaluates trained AI models and uses evaluation results to refine the model in an iterative process.

Once a model has been developed, the model can be deployed on a cloud server, and the model can be exposed to client devices through an API to provide detection/inference. Output from deployed models is then sent to the phone and transferred to a patient's EHR. Alternately (but less common), data is sent directly from the AI Cloud to a patient's EHR. Data types that are used in this process are: Time series data: blood pressure, heart rate, temperature, SP02, Spirometry data, nerve agent ecg measures. All over the time domain. Other data that can be used in this processing are Cross sectional series data: wound assessment/healing, TBI classification, chem/dx, cholinesterase (not time dependent data sets).

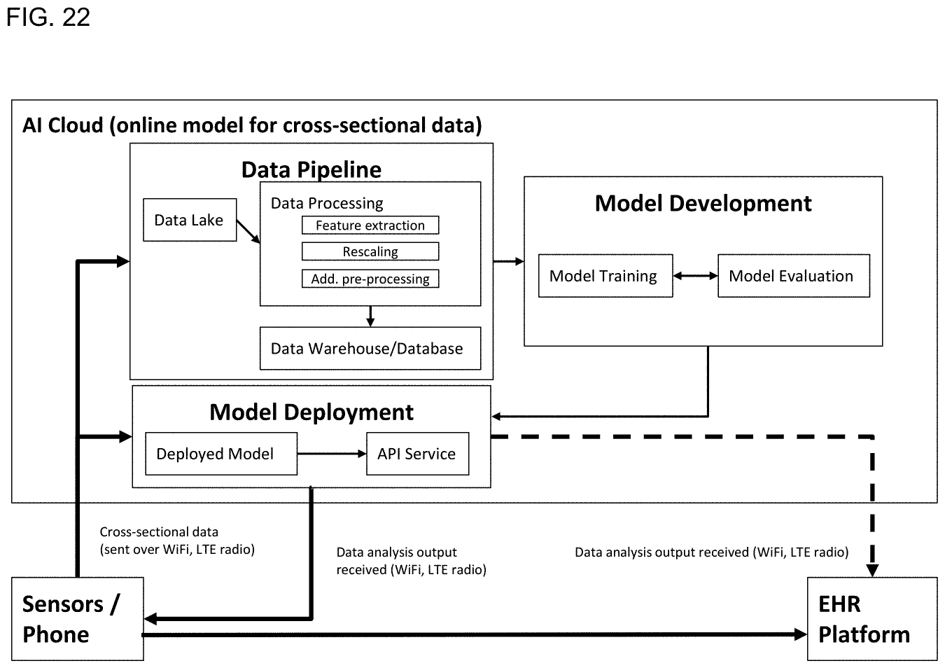

Referring to FIG. 22, for cross-sectional data, standard data processing techniques are applied as part of the data pipeline in the AI cloud, such as feature extraction, additional pre-processing, and rescaling. Feature extraction involves reducing the initial dimensionality of the raw data down to the most significant variables to improve performance and efficiency. Rescaling can then be applied to bound the domain of these variables to smaller numbers, a technique that can help models converge sooner. Model evaluation can be performed by an automated system or with human intervention, or both.

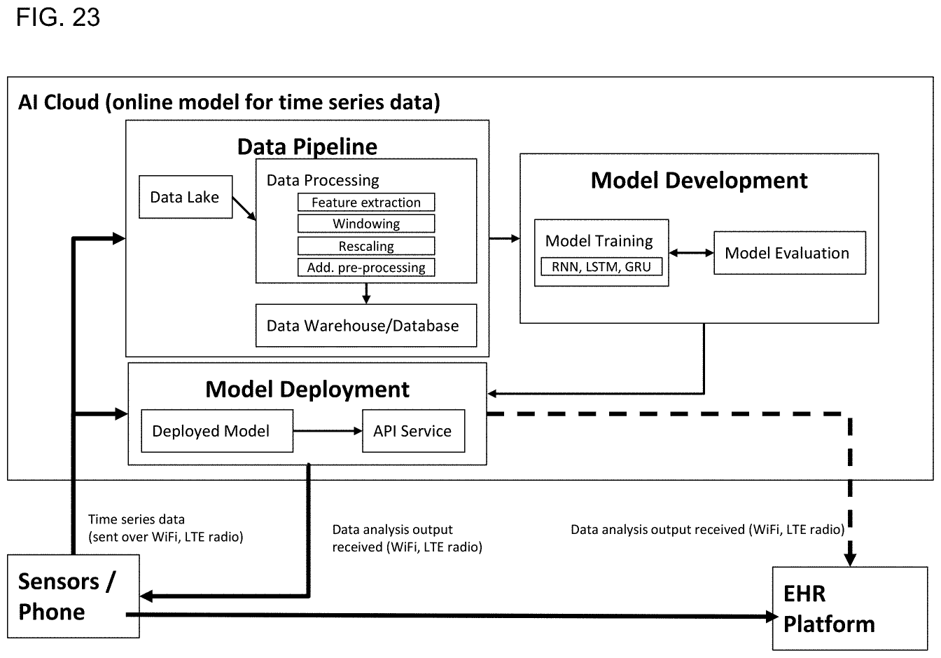

Referring to FIG. 23, time-series data involves an additional step in processing called windowing, which is determining the number of observations that form one training example. Another difference from cross-sectional data is the focus on sequence models such as recurrent neural networks (RNNs), long short-term memory (LSTMs), or gated recurrent units (GRUs) for model training.

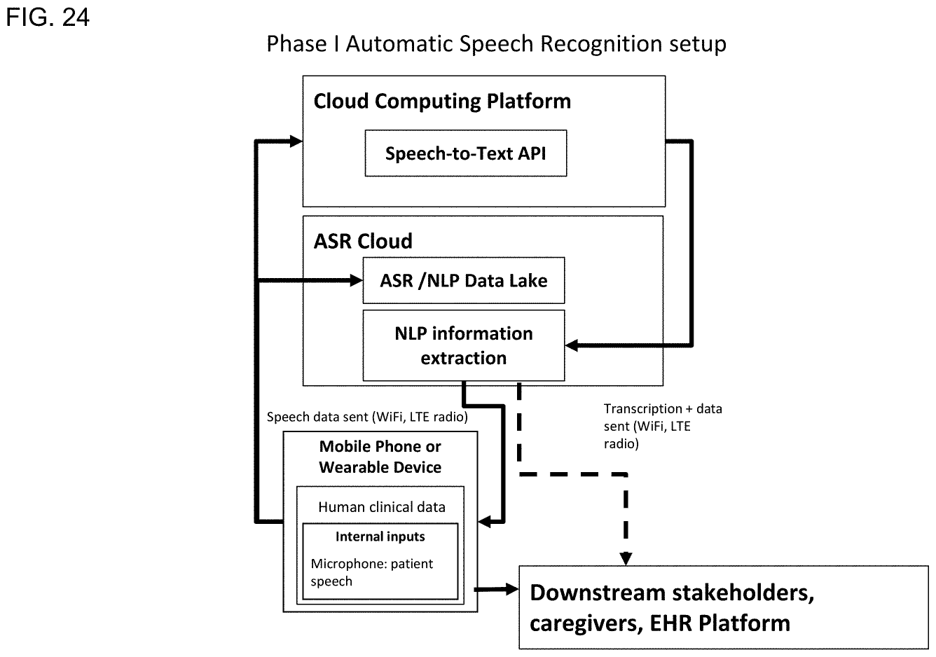

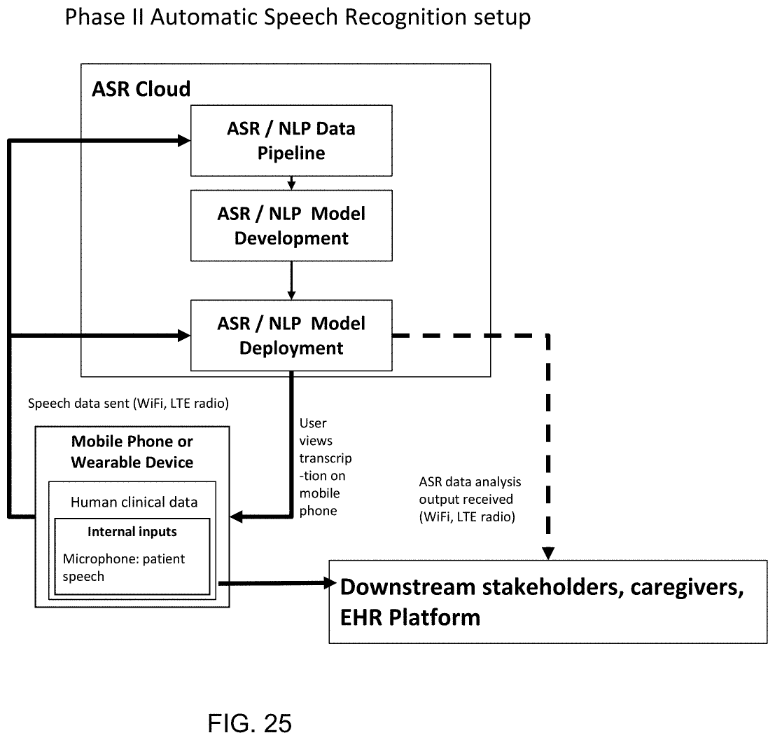

Referring to FIGS. 24-25, these diagrams illustrate the data flows involved with implementing an automatic speech recognition algorithm for the input of patient data into the CMDS system. The flow of how ASR is be performed by using existing APIs for speech-to-text transcriptions while collecting data and storing it in the cloud. In FIG. 25, the ASR data is processed and then used to train an improved, custom ASR model for deployment.

### AI/ML Medical Inventory Management Example

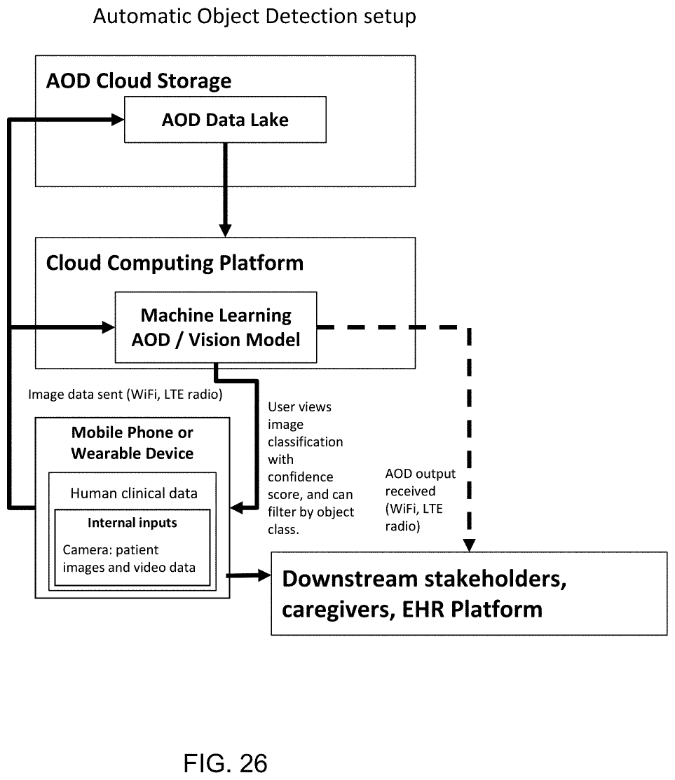

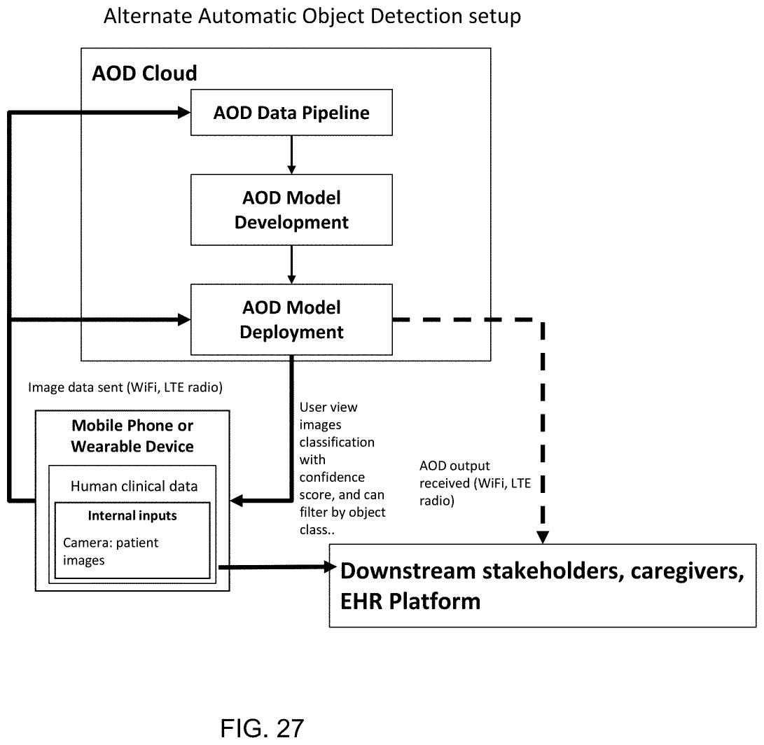

Referring to FIGS. 26-27, these diagrams illustrate the data flows involved with implementing an automatic object detection algorithm in the described CMDS system. An example of the implementation of these AI/ML methods disclosed here is an improved medical object detection model for use by combat medics to automatically track their equipment inventory as items are used while treating patients. The HMD or smartphone worn by the combat medic is loaded with a camera and a software package employing machine learning to track the use of medical items.

The system includes a robust automatic object detection (AOD) model to recognize and classify items used by Combat Medics, military clinicians, and/or civilian clinical caregivers or first responders while administering care, for a range of medical procedures. Using an mobile phone or wearable device, the AOD application detects medical items before or during use and/or after application.

This is achieved through creating an AOD training dataset and iteratively training a ML model, such as a TensorFlow Lite Edge model. Data capture for model training can be acquired by a smartphone or helmet mounted night vision cameras such as a SiOnx digital night vision camera. This is the same camera used in the Army's Integrated Visual Augmentation System (IVAS) program of record. The model may be trained using images and video data of medical items in multiple use configurations (i.e. applied to a human model and in a combat environment), under a range of lighting conditions, and from multiple angles and perspectives. The images and video data are labeled and input into a machine learning algorithm to train the model for improved performance. During use, once an object is detected with a confidence level of >75%, the application will indicate the classified object and enable users to filter by object type.

The AI/ML model can be loaded into a mobile application to track the medical equipment detected from the AOD model, to create an inventory use/management system that would provide input to medical logistics and patient documentation systems. Inventory management using a vision interface includes automatic identification, localization, classification and counting of objects through matching visual features in the detected image to those of existing stock items. The machine learning model is trained to detect medical equipment from image and video data with predefined labels. For video, metadata can be extracted at the video, shot, or frame level.

The AOD application running from a smartphone enables users to make individual predictions of medical objects from images in real-time, in addition to asynchronous batch predictions of objects within multiple previously captured images. The system makes asynchronous labeling requests for images using a machine learning model packaged with the AOD application (i.e. Android application package or APK). ML platforms (i.e., TensorFlow and PyTorch) have capabilities to export trained models to mobile devices (referred to as “edge” models), with features such as quantization that help reduce the model's size while also improving latency during object detection. We use an edge model to classify medical items in pre-captured still images to enable batch predictions for the labeling of multiple images simultaneously.

The application performs object counting using neural network libraries, such as Tensorflow and Keras. The three primary steps to run the AOD-based object counting application for inventory management include: 1) reading input image data using a computer vision library (such as OpenCV), 2) counting objects using an object counting API, which includes detecting objects (via the color recognition module) and manipulating/counting objects using their pixel locations (object counting module), and 3) generating a log file to output the object count information.

TensorFlow is a symbolic math library used for neural networks and is best suited for dataflow programming across a range of tasks. It offers multiple abstraction levels for building and training models. It is an end-to-end open-source deep learning framework with libraries and tools that facilitate building and deploying machine learning applications.

PyTorch is an open-source optimized Deep Learning tensor library based on Python and Torch and is mainly used for applications using GPUs and CPUs. It uses dynamic computation graphs and allows users to run and test portions of a code in real-time. The two main features of PyTorch are: tensor computation with strong GPU (Graphical Processing Unit) acceleration support and automatic differentiation for creating and training deep neural networks.

Keras is high-level neural network Application Programming Interface (API) written in Python. This open-source neural network library is designed to provide fast experimentation with deep neural networks, and it can run on top of CNTK (Microsoft Cognitive Toolkit), TensorFlow, and Theano (a library used for deep learning in Python).

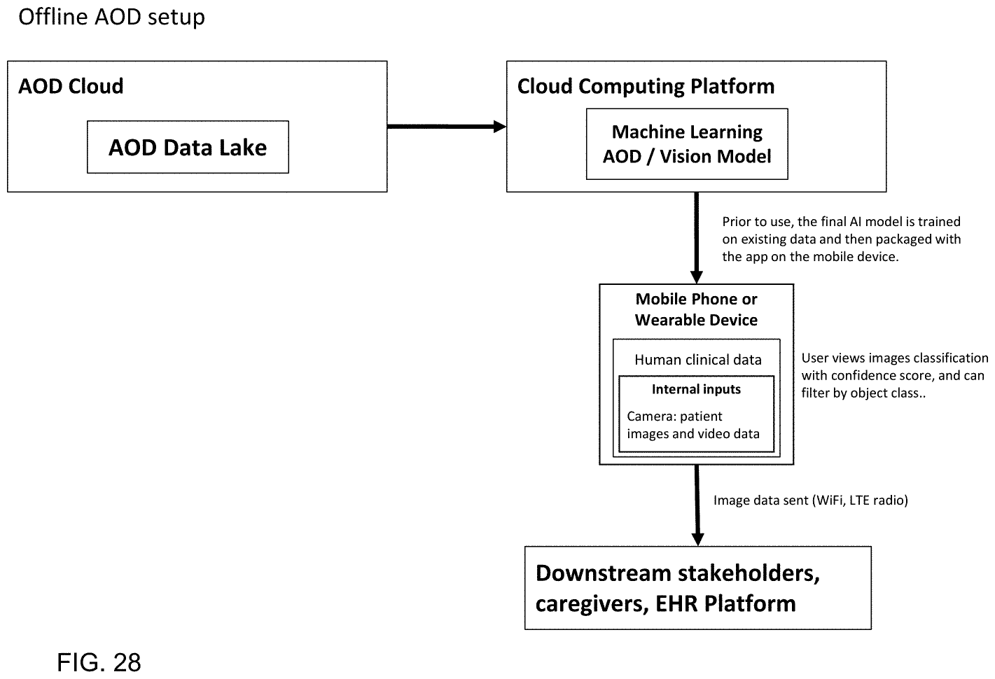

Referring to FIG. 28, an illustration of the data flows for implementing a pre-trained AOD model onto a mobile phone for offline use. This use case does not require network connectivity for the AOD model to be run, relying on the computing power available on the mobile phone or wearable device to perform the data processing for use by downstream stakeholders at a later time. The offline AOD processing may be performed on a device which is actually online but does not require communication with the cloud server.

Machine Learning Algorithms for Predictive Analytics

The systems and methods provided herein establish a framework for the development and implementation of an AI-based clinical decision support tool that combines vital sign data from wearables and static EHR data to detect a range of chemical and biological (CB) threats. For example, the systems and methods described herein can be used to predict if a patient has been exposed to a CB agent, and can be used to predict changes to a patient's health status due to the CB exposure. The predictions can include a mortality risk based on dynamic vital sign data (such as from wearable sensors described above) and on cross-sectional EHR data. Additionally, the systems and methods described herein can provide medical alerts to a caregiver, such as flagging vital sign values that are out of a normal range (e.g., HR, BP, Temp, SpO2, etc.).

The framework described herein can have wide applicability for disease detection and mortality risk assessment. For example, the systems and methods described herein can be used to detect disease in a patient, such as detecting whether a patient is infected with the flu or with COVID-19. Additionally, the methods and systems described herein can be used to assess the mortality risk of a patient diagnosed with disease, such as COVID-19 mortality risk. As described above, the machine learning models herein can provide a predictive output based on a combination of data inputs, including streaming vital sign or time-series data and cross-sectional EHR data.

Additional application areas for detection and/or mortality risk the framework described herein can include sepsis/septic shock detection and mortality risk, acute respiratory failure and mortality risk, blast pressure and head impact (TBI/concussion detection) and mortality risk, heat exposure and mortality risk, ocular injury and protection, physiological chemical toxicity/chemical burn assessment and mortality risk, and musculoskeletal injury and mortality risk.

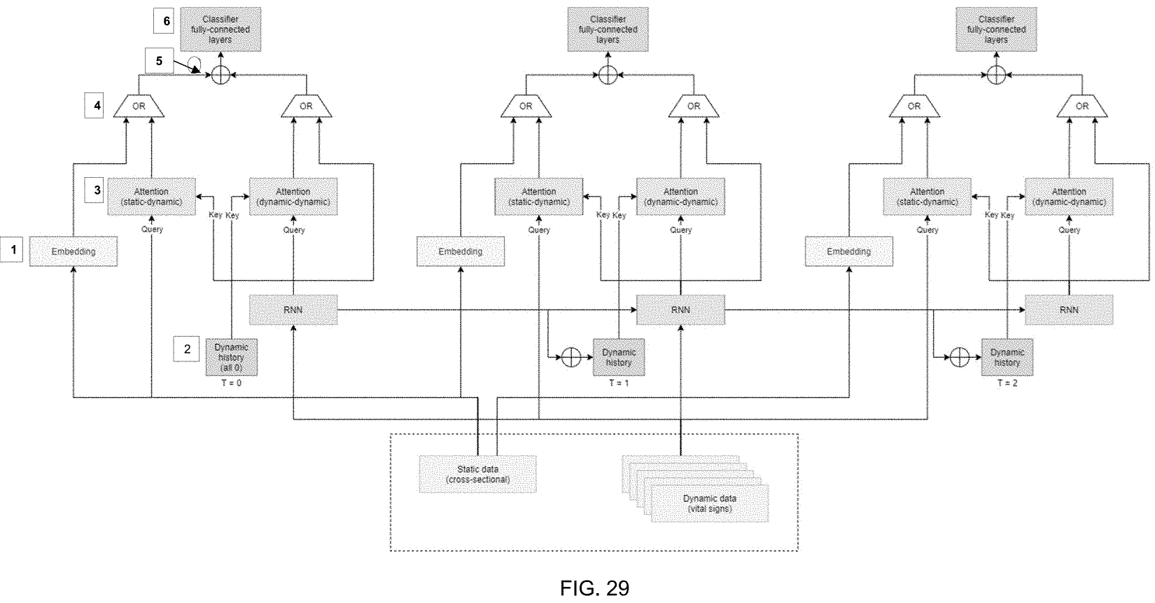

Referring to FIG. 29, the AI-based clinical decision support system for mortality risk prediction due to CB exposure uses a RNN voting ensemble of sequential models. The model uses either an LSTM or GRU recurrent unit with optional stacked layers. To include the cross-sectional static data in the RNN model, the model includes a linear embedding layer 01 to embed the included features in a higher-dimensional space. The RNN output hidden states at each time step are stored in a dynamic history vector, initialized with all zeros 02. This dynamic history vector is used downstream to compute the dynamic-dynamic attention score at each time step. The computation of two different attention scores that may be enabled by the practitioner. The static-dynamic attention scores (one score per static feature) 03 represent how much each feature “attends to” the current RNN hidden state, or the current time step's dynamic measurements. The dynamic-dynamic attention scores (one score per node in the hidden state) represent how much the current time step's dynamic measurement. “attends to” each of the previous time steps' hidden representations. The model allows one of two inputs to progress to the next stage in the model pipeline, depending on which attention mechanisms the practitioner enables 04. If static-dynamic attention is enabled, the attention scores for the static features progress to the next stage. Otherwise, the linear embedding layer output is used. If dynamic-dynamic attention is enabled, the attention scores for the current time step's hidden representation progresses to the next stage. Otherwise, the current time step's hidden representation is used. Finally, the static and dynamic vectors are concatenated 05 and passed as input into fully-connected classification layers 06 to obtain the model prediction for the current time step.

Prior Bias. There is an option for the final fully-connected layer to incorporate the prior label probabilities in order to bias the prediction generated at each time step and better account for the class imbalance. This sets the bias on the logits such that the neural network predicts the probability of the positive class (p) at initialization for imbalanced datasets. In other words, the output is p after the sigmoid and χ denotes the desired input to the sigmoid, and therefore the desired bias value:

$$p = \frac{1}{1 + {e}^{- x}} \\ x = - log ( \frac{1 - p}{p \vert} )$$

Focal Loss. Focal loss was implemented to add a factor (1−pₜ)^{y} to the standard cross entropy criterion and effectively reduce the relative loss for well-classified examples, putting more focus on hard, misclassified examples. With this notation, pₜ=p if the label is positive and pₜ=1−p if the label is negative, where p is the model's estimated probability for the class with label y=1. Therefore, CE(p, y)=CE(pₜ)=— log(pₜ) where CE is the cross-entropy loss for binary classification and Focal Loss is defined as FL(pₜ)=−(1−pₜ)^{y} log(pₜ).

Attention is used to find the similarity between a “key” and a “query” by computing the dot product. It is used to identify which features or information in parts of the network that another part of the network “attends to” or finds most relevant to classification. More specifically, in sequential models it is used to identify important time steps by comparing the hidden states of the network. An attention score is computed by using the dot product, or a variation of the dot product with a linear transformation. These scores are used to weight the hidden states in a weighted average of the hidden states such that more important time steps contribute more to the prediction.

To compute the attention between the static and dynamic variables, the static data is the query and the output from the RNN is the key. Similarly, to compute the attention between the current dynamic data and the history of dynamic data, the current data is the query and the history is the key. If both attention mechanisms are used, the features passed to the fully-connected layers consist of both static-to-dynamic and dynamic-to-dynamic attention context vectors. If only the static-dynamic mechanism is used, the features consist only of the static-to-dynamic attention vector. Finally, if only the dynamic-dynamic mechanism is used, the features consist of the embedded static data and the dynamic-to-dynamic attention vector.

Calibration. The outputs from the neural network, or logits, may be scaled between 0 and 1 with a sigmoid function and a threshold is applied to obtain predictions.

Ensemble approaches have yielded favorable results while addressing imbalanced data sets by aggregating the predictions from multiple weak learners/different models.

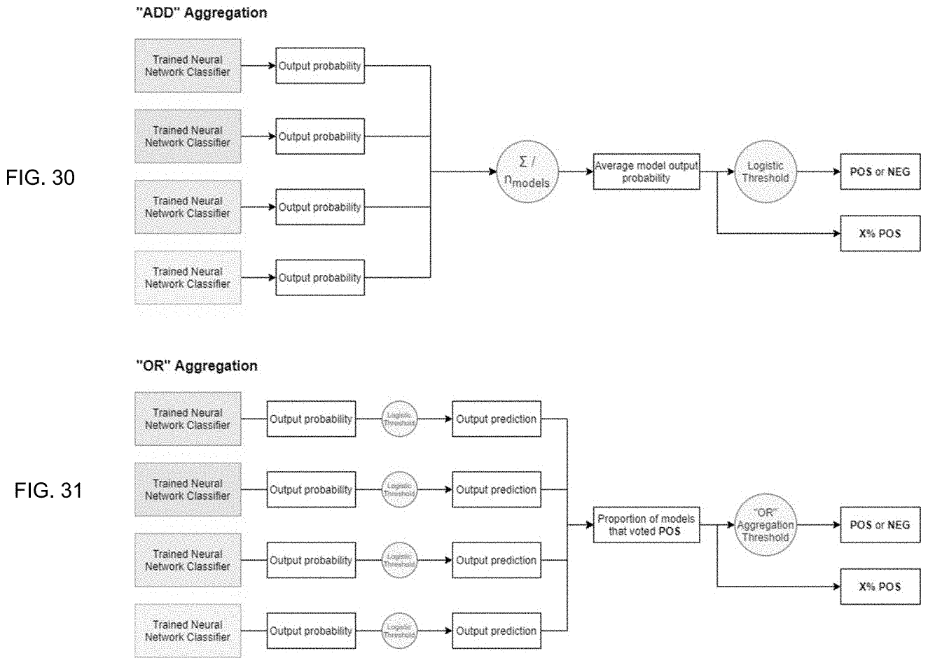

Referring to FIGS. 30-31, the RNN ensemble method utilizes a voting system that heuristically chooses the “most different” models (i.e. the ones that make the most different predictions) in order to encourage model diversity without penalizing the results. There are many ways of aggregating predictions among voting models. The approaches in this embodiment include “ADD” and “OR” aggregation. This improves the overall ensemble's sensitivity while reducing the variability of evaluation metrics across cross-validation folds.

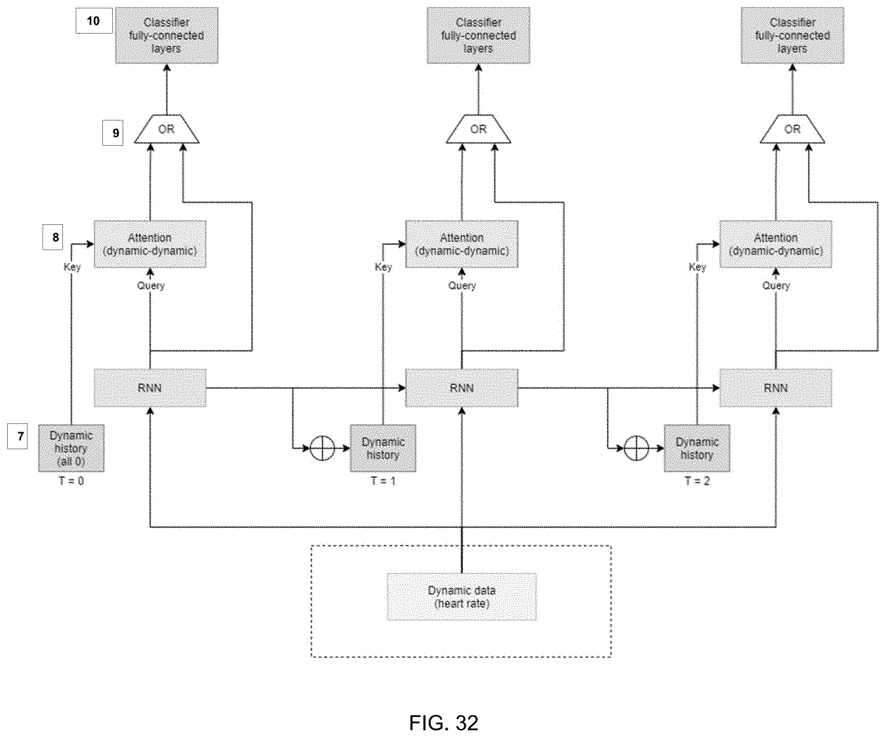

Referring to FIG. 32, the system includes a sequential model for chemical and biological (CB) exposure prediction, based on heart rate time-series data (averaged hourly). The sequence model uses either a Long Short-Term Memory (LSTM) or Gated Recurrent Units (GRU) with optional stacked layers. A unidirectional RNN was used in this study due to the causal nature of the time-series clinical data. The model uses attention mechanisms between each current time step measurement and the history of past time step measurements for a given patient. This adds a layer of interpretability to the model and improves performance by allowing the model to “focus” on specific time steps that are influential to the model's prediction. After the RNN output is a series of fully-connected layers, where the number of nodes in each additional layer equals half the number of nodes in the previous layer.

Referring to FIG. 32, the RNN output hidden states at each time step are stored in a dynamic history vector, initialized with all zeros 07. This dynamic history vector is used downstream to compute the dynamic-dynamic attention score at each time step. The computation of the dynamic-dynamic attention scores may be enabled by the practitioner 08. The dynamic-dynamic attention scores (one score per node in the hidden state) represent how much the current time step's dynamic measurement “attends to” each of the previous time steps' hidden representations. The model allows one of two inputs to progress to the next stage in the model pipeline depending on which attention mechanisms the practitioner enables 09. If dynamic-dynamic attention is enabled, the attention scores for the current time step's hidden representation progresses to the next stage. Otherwise, the current time step's hidden representation is used. Finally, the static and dynamic vectors are passed as input into fully-connected classification layers 10 to obtain the model prediction for the current time step.

Training the CB detection model includes the use of Binary Cross-Entropy (BCE) as the criterion and optimized using the Adam optimizer (weight decay=0.000). Two classifying fully-connected layers after the RNN output are used, with a dropout probability of 0.15 and the prior probability of the label to condition the output. Gradient clipping was implemented (0.25) and the logistic threshold was 0.5. The logistic threshold is the lower bound for predicted probabilities by the model to be considered a prediction for the positive class. For instance, for predicted probabilities greater than or equal to 0.5, the patient is considered positive for a condition; and for probabilities less than 0.5, the patient is negative. Model instances were trained in a deterministic manner, with a training seed of 42 and a cross-validation seed of 15.

The following metrics are used to evaluate model performance: Accuracy measures the number of correct predictions over the total number of cases; F1 score is the harmonic mean of the model's precision and recall; the macro F1 score is used to compute the metric for each label and find the unweighted mean, such that this score does not account for the label imbalance in the data set; Sensitivity measures the capacity to correctly predict a model outcome, such as mortality (also known a recall). It is equal to the proportion of the number of true positives to the total number of positive instances; Specificity measures the capacity to correctly predict an outcome (i.e. mortality). It is equal to the proportion of the number of true negatives to the total number of negative instances; Area Under the Curve (AUC) is the probability of a random example with a positive label to get a higher score than a random example with a negative label. Plots the false positive rate (FPR) on the x-axis (also equal to 1−specificity) and the true positive rate (TPR) on the y-axis (equal to sensitivity); Global Performance Evaluation is the average metric over all time steps in a patient's sequence for all patients; Daily Performance Evaluation is the daily performance metrics computed from the outcome date (align all predictions on the right/by the outcome date); Patient Population Performance Evaluation is the evaluation metrics computed across all patient's final predicted outcome (i.e. evaluation of the model's prediction for each patient's final time step compared to ground truth discharge or mortality).

ML model metrics include validation metrics averaged over 10-fold cross-validation splits and performance metrics based on a held-out test set. In addition to the average validation performance metrics, the standard deviation of these performance metrics is important in evaluating the robustness of the model. The ideal model will have a low standard deviation for the performance metrics across the 10 iterations of models trained.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Thus, although specific embodiments have been illustrated and described herein, any arrangement calculated to achieve the same purpose may be substituted for the specific embodiments shown.

This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method for treating a patient, comprising the steps of:
obtaining cross-sectional data related to a patient;
capturing time-series physiological data from the patient;
inputting the cross-sectional data and the time-series physiological data into a trained machine learning model; and
outputting a patient score from the machine learning model that provides an assessment of the patient's health.

2. The method of claim 1, wherein the patient score comprises an infectious disease diagnosis.

3. The method of claim 1, wherein the patient score comprises an indication of chemical-biological (CB) exposure.

4. The method of claim 1, wherein the patient score comprises a mortality assessment.

5. The method of claim 1, wherein the machine learning models for infectious disease diagnosis, CB exposure detection, and mortality risk prediction due to CB exposure use a RNN voting ensemble of sequential models. (or RNN voting ensemble models)

6. The method of claim 1, wherein clinical data entry modes include manual data entry, automatic/passive clinical data capture from wearable sensors, voice-driven automatic speech recognition, and automatic object detection from image and video data.

7. The method of claim 1, wherein the cross-sectional data is obtained from the patient's electronic health record (EHR).

8. The method of claim 1, wherein the cross-sectional data comprises an assessment of the patient from a medical provider.

9. The method of claim 1, wherein the cross-sectional data comprises patient medical history.

10. The method of claim 1, wherein the time-series physiological data is captured in real-time by sensors worn by the patient.

11. The method of claim 1, wherein the time-series physiological data is selected from the group consisting of activity, activity-based energy expenditure (AEE), accelerometry-based total daily energy expenditure (TDEE), arterial oxygen saturation (SaO2), arteriovenous oxygen difference (a-vO2), blood glucose level, cardiac waveform data, capnography (CO2 concentration), core body temp temperature (CBTemp), electrocardiogram (ECG or EKG), electrodermal activity (EDA), electroencephalograms (EEG), end-tidal CO2, extremity temperature, galvanic skin response (GSR) sensor for measuring skins electrical properties (conductance, resistance, impedance, capacitance), heart rate (HR), heart rate variability (HRV), hydration levels, Nerve agent time series data (ECG measures), motion, peripheral oxygen saturation (SpO2), Pulse Oximetry, photoplethysmogram (PPG), plethysmography, respiration rate (Resp or RR), skin temperature (Skin Temp), systolic, mean, and/or diastolic blood pressure (BP), spirometry data for pre- and post-particulate exposure, and time-series data for language classification.

12. The method of claim 1, further comprising storing the time-series physiological data and the cross-sectional data on an electronic device worn by the patient.

13. The method of claim 1, wherein the trained machine learning model is developed and stored on an electronic device worn by the patient.

14. The method of claim 1, wherein the trained machine learning model is developed and stored on a cloud computing server.

15. A system configured to provide medical treatment to a patient, comprising:
a personal computing device configured to record patient information and prior treatment information;
a sensor unit configured to be worn by the patient and to record patient physiological measurements;
an electronic data tag configured to store the patient physiological measurements, the patient information, and the prior treatment information; and
a trained machine learning model configured provide a patient score that provides an assessment of the patient's health based on the patient physiological measurements, the patient information, and the prior treatment information.

16. The system of claim 15, wherein the personal computing device comprises a head-mounted display (HMD).

17. The system of claim 15, wherein the personal computing device comprises a smartphone.

18. The system of claim 15, wherein the sensor unit comprises a fabric sleeve with integrated sensors.

19. The system of claim 15, wherein the electronic data tag and sensor unit are configured to communicate by a wireless connection.

20. The system of claim 15, wherein the personal computing device is configured to record patient information with a verbal input from a caregiver.

21. The system of claim 15, wherein the patient score is displayed on the electronic data tag.

22. The system of claim 15, wherein the patient score is displayed on the personal computing device.

23. A non-transitory computing device readable medium having instructions stored thereon for determining a patient score that provides an assessment of the patient's health, wherein the instructions are executable by a processor to cause a computing device to:
obtain cross-sectional data related to a patient;
capture time-series physiological data from the patient;
input the cross-sectional data and the time-series physiological data into a trained machine learning model; and
output a patient score from the machine learning model that provides an assessment of the patient's health.
